(19) United States
(12) Patent Application Publication
(10) Pub. No.: US 2016/0206543 A1
(43) Pub. Date: Jul. 21, 2016

(54) SYNERGISTIC COMPOSITIONS AND METHODS FOR MITIGATING SKIN IRRITATION AND ENHANCING SKIN BARRIER FUNCTION

(71) Applicant: GOJO INDUSTRIES, INC., Akron, OH (US)

(72) Inventors: Jessica R. Tittl, Chagrin Falls, OH (US); Christopher A. Wolf, Ashland, OH (US)

(21) Appl. No.: 15/086,487

(22) Filed: Mar. 31, 2016

Related U.S. Application Data
(63) Continuation-in-part of application No. 14/750,241, filed on Jun. 25, 2015.
(60) Provisional application No. 62/140,542, filed on Mar. 31, 2015.
(60) Provisional application No. 62/258,716, filed on Nov. 23, 2015.
(60) Provisional application No. 62/021,310, filed on Jul. 7, 2014.
(60) Provisional application No. 62/077,349, filed on Nov. 10, 2014.

(51) Int. Cl. A61K 8/64; A61Q 19/00; A61K 8/37; A61K 8/92; A61K 8/02

(52) U.S. Cl. CPC A61K 8/64; A61K 8/922; A61K 8/0295; A61K 8/375; A61Q 19/00; A61K 2800/596; A61K 2800/49; A61K 2800/74

(57) ABSTRACT

Methods and compositions are provided for mitigating the irritation of mammalian skin cells, protecting cell viability and/or enhancing cell-cell junction, thus improving skin barrier function. The compositions contain combinations of acetyl hexapeptides and a cosmetically and/or pharmaceutically acceptable carrier. Carriers that lend a liquid crystalline aspect to the compositions are beneficial.

### TECHNICAL FIELD

One or more embodiments of the present invention provide methods and compositions for improving skin barrier function and skin hydration. More particularly, it relates to compositions containing synergistic combinations of acetyl hexapeptides in a cosmetically or pharmaceutically acceptable carrier. Compositions of the invention reduce inflammation and protect and heal damaged skin.

### BACKGROUND OF THE INVENTION

Many ingredients in skin care and cosmetic products can cause skin irritation. Surfactants such as sodium lauryl sulfate (SLS) are known to be skin irritants. Retinoid and its derivatives, can cause severe local irritation manifested as mild erythema and stratum corneum peeling of the skin. Topical or systemic use of some skin cleansers and disinfectants is linked to skin irritation.

Ingredients such as benzoyl peroxide, alpha-hydroxyl acids and derivatives thereof, salicylic acid, natural plant extracts, sunscreen actives, urea, and preservatives are also known to cause external skin irritations. Furthermore, skin irritations may be caused by inherent disease conditions such as acne, rosacea, atopic dermatitis, and other disease states. Typical approaches to reduce irritation include reducing the concentration of the inflammatory ingredient, use of alternatives or formulation/delivery approaches, such as encapsulation, controlled release, compartmentalization, inclusion of non-irritating excipients. None of the above has successfully reduced irritation while retaining efficacy. As a result, there is a need for anti-irritant substances to mitigate external skin irritations, or irritations caused by inherent skin conditions.

Skin exposure to water and typical cleansers may have a negative effect on the stratum corneum (SC) structure and function. Effects include disruption of the lipid bilayer architecture to create defects or holes in the barrier. As a result, the barrier becomes more permeable, allowing irritants and microorganisms to penetrate into and through the uppermost layers of the skin. In cases of severe hand irritation, cracks or fissures (with or without bleeding) may develop indicating damage to the dermis. The skin's response to these damaging effects is immediate, but the accelerated efforts to repair the barrier and generate new stratum corneum leads to imperfect architecture, when compared to stratum corneum that is formed during the normal course of SC replacement. The rapidly-produced SC has poor water binding properties, leading to insufficient skin moisture and inadequate desquamation.

Under normal conditions, there is also a constant loss of SC cells, as individual units from the surface of the skin, and new cells move from the bottom of the SC to the surface, generally over a period of about 14 days. When skin moisture is too low, the SC cells come off of the skin surface as clumps of cells, observed as dry scales. There are ingredients in most skin care and cosmetic products that accelerate the loss of SC cells, by affecting cell viability and/or by modulating epidermal proliferation. There is a substantial need for products that protect cell viability and proliferation, while at the same time decreasing irritation of the skin cells caused by exposure to water and typical cleansers.

Acetyl hexapeptides such as acetyl hexapeptide-3 have been employed for properties including anti-wrinkle, collagen boosting, anti-aging, and relaxing of facial tension. Acetyl hexapeptide-3 is said to be non-irritating. Some peptides have been described as capable of stimulating collagen synthesis, and increasing the hydration of the skin.

However, specific combinations of acetyl hexapeptides have not heretofore been described as providing synergistically enhanced reductions in skin irritation, nor have they been described as enhancing skin barrier function and the hydrating efficacy of lotions.

Pressure sores, sometimes referred to as bed sores or decubitus ulcers, may develop if a person spends a significant amount of time in a wheelchair, regular chair, or bed. The likelihood of developing pressure sores is exacerbated if the person is incontinent, is elderly, has received radiation therapy, and/or has not been eating well. Conventional incontinence pads and bandages can make skin problems worse. Although they may keep bedding and clothing cleaner, these products can trap urine, feces, or other possible irritants, keeping them in constant contact with the skin. Over time, the skin may break down, and ulcers are likely to develop.

Healthcare experts have noted that pressure sores are easier to prevent than to treat. Conventional treatment includes multiple steps of applying multiple products for cleaning, wound healing, moisturization/hydration of the skin, and barrier protection.

Many of the current treatments fail to provide adequate skin protection. Thus, they fail to eliminate further skin irritation and facilitate the natural healing process. Others fail to resist removal when contacted by clothing and body fluids. Still others fail to provide acceptable results within a reasonable time period. Some ointments are difficult to apply and spread on the damaged skin. There exists a need in the art for a topical cream formulation that is easy to apply, physically stable (i.e. without phase separation), chemically stable, that is well tolerated by and suitable for use in individuals with sensitive, reactive, easily irritated or damaged skin.

### BRIEF DESCRIPTION OF THE DRAWINGS

FIG. 1 is a graphical representation of the effect of compositions containing oligopeptides on the irritation response of cells treated with known irritants, as quantified by measuring IL-8 secretion.

FIG. 2 is a graphical representation of the reduction of IL-8 secretion for test samples, compared to Control B, which contained no oligopeptide.

FIG. 3 is a graphical representation of the effect of oligopeptides on cell binding, as measured by Real-Time Quantitative Reverse Transcription Polymerase Chain Reaction (qRT-PCR) for desmoglein (DSG1).

FIG. 4 is a graphical representation of the effect of oligopeptides on cell binding, as measured by qRT-PCR for democollin (DSC3).

FIG. 5 is a graphical representation of the effect of various compositions on the irritation response of cells treated with known irritants, as quantified by measuring IL-8 secretion, and on cell viability.

FIG. 6 is a graphical representation of Involucrin Expression.

FIG. 7 is a graphical representation of PPARδ expression.

FIG. 8 is a graphical representation of ABCA12 expression.

FIG. 9 is a graphical representation of DSC1 expression.

FIG. 10 is a graphical representation of APQ3 expression.

FIG. 11 is a graphical representation of yield stress point for compositions according to the present invention and comparative samples.

FIG. 12 is a graphical representation of thixotropic behavior for compositions according to the present invention and comparative samples.

### DETAILED DESCRIPTION OF ILLUSTRATIVE EMBODIMENTS

In one or more embodiments, compositions of the present invention include a combination of at least two oligopeptides in a cosmetically or pharmaceutically acceptable carrier.

Generally, oligopeptides are short chains of amino acid moieties linked by amide bonds. Any combination of amino acids may be included.

In one or more embodiments, the oligopeptides include 12 amino acid moieties or less, in other embodiments, 10 amino acid moieties or less, and in other embodiments, 8 amino acid moieties or less.

In one or more embodiments, the peptides include at least 5 amino acid moieties. In one or more embodiments, the peptide includes at least 6 amino acid moieties. In one or more embodiments, the peptides include from 5 to 6 amino acid moieties. Longer peptides are believed to have at least the potential for causing undesirable responses on application to the human body. In one or more embodiments, the stereochemistry of at least one of the amino acids of the peptide is L−.

In other embodiments, the oligopeptide may be identified by an INCI (International Nomenclature of Cosmetic Ingredients) name, such as acetyl hexapeptide followed by a numerical designation. INCI names for suitable oligopeptides include Polmitoyl Oligopeptide, Glycerin (and) Aqua (and) Myristoyl Pentapeptide-17, Aqua (and) Butylene Glycol (and) Oryza Sativa (Rice) Bran Extract (and) Boswellia Serrata Extract (and) Honey Extract (and) Oligopeptide-10 (and) Phenoxyethanol (and) Sodium Benzoate, Oligopeptide-1, rh-Oligopeptide-1, rh-Oligopeptide-1 (and) Mannitol (and) Glycerin (and) EDTA (and) Methylparaben (and) Ethylparaben, Butylene Glycol (and) Hydrogenated Lecithin (and) Sodium Oleate (and) Oligopeptide-68, Hydrolyzed Wheat Protein (and) Palmitoyl Decapeptide-21 (and) Decapaptide-22 (and) Oligopeptide-78 (and) Zinc Palmitoyl Nonapeptide-14, Hydrolyzed Wheat Protein (and) Palmitoyl Decapeptide-21 (and) Decapeptide-22 (and) Oligopeptide-78 (and) Zinc Palmitoyl Nonapeptide-14, and Aqua (and) Rahnella/Soy Protein Ferment (and) Glycerin (and) Butylene Glycol (and) Glyceryl Acrylate/Acrylic Acid Copolymer (and) Polysorbate 20 (and) Palmitoyl Oligopeptide.

Oligopeptides are further described in U.S. Published Patent Application No. 2014/0120141 A1, and co-pending U.S. application No. 62/021,310, both of which are hereby incorporated by reference.

In one or more embodiments, compositions of the present invention include at least two acetyl hexapeptides. In certain embodiments, a synergistic effect is seen when two or more oligopeptides are combined. In other words, the reduction in irritancy when two or more oligopeptides are combined is greater than just an additive effect. In one or more embodiments, the positive effect on cell regeneration is more than the sum of the improvement that is seen with equivalent amounts of the individual components.

Thus, in one or more embodiments, compositions of the present invention comprise two or more oligopeptides. In one or more embodiments, compositions of the present invention comprise two or more acetyl hexapeptides. In one or more embodiments, compositions of the present invention comprise two or more acetyl hexapeptides, and at least one of the acetyl hexapeptides is selected from acetyl hexapeptide-38 and acetyl hexapeptide-46. In one or more embodiments, compositions of the present invention comprise acetyl hexapeptide-38 and acetyl hexapeptide-46.

Generally, hexapeptides contain six amino acid moieties. Some non-limiting examples of acetyl hexapeptides are acetyl hexapeptide-1, acetyl hexapeptide-7, acetyl hexapeptide-8, acetyl hexapeptide-19, acetyl hexapeptide-20, acetyl hexapeptide-22, acetyl hexapeptide-24, acetyl hexapeptide-30, acetyl hexapeptide-31, acetyl hexapeptide-37, acetyl hexapeptide-38, acetyl hexapeptide-39, acetyl hexapeptide-46, and acetyl hexapeptide-49.

Acetyl Hexapeptide-1 is reaction product of alanine, arginine, histidine, leucine, phenylalanine and tryptophane hexapeptide with acetic acid. Acetyl hexapeptide-1 is commercially available, for example from Lucas Meyer Cosmetics as a blend with glycerin and water and dextran under the tradename Melitane®.

Acetyl hexapeptide-8 is commercially available, for example from Theraderm Clinical Skin Care under the tradename Argireline®.

Acetyl hexapeptide-30 is commercially available, for example from Lipotec LLC under the tradename Inyline®.

Acetyl hexapeptide-38 is commercially available, for example from Lipotec LLC as a blend with butylene glycol and water, under the tradename Adifyline™.

Acetyl hexapeptide-39 is commercially available, for example from Lipotec LLC under the tradename Silusyne®.

Acetyl hexapeptide-46 is commercially available, for example from Lipotec LLC as a blend with butylene glycol, water and citric acid, under the tradename Delisens®.

Other acetyl hexapeptides are also commercially available.

In one or more embodiments, the composition includes acetyl hexapeptide-38 and acetyl hexapeptide-46.

In one or more embodiments, compositions of the present invention include at least one pentapeptide. Generally, pentapeptides contain five amino acid moieties. Some non-limiting examples of pentapeptides include acetyl pentapeptide-1, pamitoyl pentapeptide-3, pamitoyl pentapeptide-4, and myristoyl pentapeptide-17. In one or more embodiments, compositions of the present invention comprise pamitoyl pentapeptide-3. In one or more embodiments, compositions of the present invention comprise pamitoyl pentapeptide-4. In one or more embodiments, compositions of the present invention comprise myristoyl pentapeptide-17.

Acetyl pentapeptide-1 is commercially available, for example from Spec Chem Ind. Under the tradename SpecPed SC-AP1. Pamitoyl pentapeptide-3 is commercially available, for example from Spec Chem Ind. Under the tradename SpecPed SC-PP3. Myristoyl pentapeptide-17 is commercially available, for example from Spec Chem Ind. Under the tradename SpecPed SC-MP17.

The cosmetically or pharmaceutically effective amount of the peptides of the invention which should be administered, as well as their dosage, will depend on numerous factors, including age, state of the patient, the nature or severity of the condition, disorder or disease to be treated and/or cared for, the route and frequency of administration and of the particular nature of the peptides to be used.

In one or more embodiments, compositions of the present invention comprise at least an effective amount of the oligopeptide, wherein an effective amount is the amount that mitigates skin irritation, enhances skin conditioning, enhances the skin barrier function, provides hydration, or enhances healing of damaged skin, when compared to the same composition but not containing any oligopeptide. In one or more embodiments, an effective amount of each oligopeptide is at least about 0.06 parts per million by weight (ppm), based upon the total weight of the composition. In other embodiments, an effective amount is at least about 0.10 ppm, in other embodiments at least about 0.12 ppm, based upon the total weight of the composition.

In one or more embodiments, the total amount of oligopeptides in the composition is at least about 0.12 ppm, in other embodiments, at least about 0.15 ppm, in other embodiments, at least about 0.17 ppm, in other embodiments, at least about 0.2 ppm, in other embodiments, at least about 0.25 ppm, based upon the total weight of the composition.

In one or more embodiments, the composition comprises about 100 ppm or less of each oligopeptide, in other embodiments, about 50 ppm or less, in other embodiments, about 25 ppm or less, in other embodiments, about 10 ppm or less, in other embodiments, about 7.5 ppm or less, in other embodiments, about 5 ppm or less, in other embodiments, about 2.5 ppm or less, in other embodiments, about 2 ppm or less, in other embodiments, about 1.5 ppm or less, in other embodiments, about 1 ppm or less, based upon the total weight of the composition.

The total amount of oligopeptides in the composition is not particularly limited. Advantageously, due to the synergistic enhancement in efficacy that is obtained by combining two or more oligopeptides, the total amount of oligopeptide may be reduced. In one or more embodiments, the composition comprises a total of about 1000 ppm or less of oligopeptides, in other embodiments, about 800 ppm or less, in other embodiments, about 600 ppm or less, in other embodiments, about 500 ppm or less, in other embodiments, about 250 ppm or less, in other embodiments, about 200 ppm or less, in other embodiments, about 150 ppm or less, in other embodiments, about 100 ppm or less, in other embodiments, about 75 ppm or less, in other embodiments, about 50 ppm or less, in other embodiments, about 25 ppm or less, in other embodiments, about 20 ppm or less, in other embodiments, about 10 ppm or less, based upon the total weight of the composition.

In one or more embodiments, the composition comprises from about 0.06 to about 100 ppm of each oligopeptide, based upon the total weight of the composition. In one or more embodiments, the composition comprises from about 0.08 to about 50 ppm of each oligopeptide, based upon the total weight of the composition. In one or more embodiments, the composition comprises from about 0.1 to about 30 ppm of each oligopeptide, based upon the total weight of the composition. In one or more embodiments, the composition comprises from about 0.5 to about 25 ppm of oligopeptide, based upon the total weight of the composition.

The oligopeptides of this invention may have variable solubility in water. Water-soluble oligopeptides may be incorporated directly into aqueous compositions. Water-insoluble oligopeptides and those with limited water solubility may be solubilized in cosmetically or pharmaceutically acceptable solvents such as and not restricted to, ethanol, propanol, isopropanol, propylene glycol, glycerin, butylene glycol or polyethylene glycol or any combination thereof.

In one or more embodiments, the oligopeptide may be added to the composition as a solution or emulsion. In other words, the oligopeptide may be premixed with a diluent, and optionally one or more other ingredients, to form an oligopeptide solution or emulsion, with the proviso that the diluent does not deleteriously affect the beneficial properties of the composition.

Examples of diluents include water, alcohol, or blends of water and other diluents such as glycols, ketones, linear and/or cyclic hydrocarbons, triglycerides, carbonates, silicones, alkenes, esters such as acetates, benzoates, fatty esters, glyceryl esters, ethers, amides, polyethylene glycols, PEG/PPG copolymers, inorganic salt solutions such as saline, and mixtures thereof. It will be understood that, when the oligopeptide is premixed to form an oligopeptide solution or emulsion, the amount of solution or emulsion that is added to the composition is selected so that the amount of oligopeptide falls within the ranges set forth hereinabove.

In one or more embodiments, the cosmetically or pharmaceutically acceptable carrier may be prepared according to convention known to persons skilled in the art. For example, the formulation of cosmetic and pharmaceutical lotion compositions is described in “Harry's Cosmeticology,” Eighth edition, (2000), Harry, Ralph Gordon, Reiger, Martin M., ed. Chemical Publishing Company, which is hereby incorporated by reference.

The form of the cosmetically or pharmaceutically acceptable carrier is not particularly limited, and compositions of the present invention may be formulated as liquids, lotions, creams, gels, foams, salves, suspensions, emulsions, and the like. Suitable formulations are described, for example, in U.S. Pat. Nos. 5,980,970, 8,105,616, U.S. Patent Application Publication Nos. 2006/0159649 A1, 2009/0041697 A1, 2014/0011894 A1, and 2014/0328769 A1, all of which are incorporated by reference herein.

A wide variety of delivery vehicles may be employed for compositions according to the present invention, including pads, bandages, patches, sticks, aerosol dispensers, pump sprays, trigger sprays, canisters, and disposable absorbent articles.

In one or more embodiments, the compositions of the present invention may be formulated as lotions. As is known in the art, lotions include oil-in-water emulsions as well as water-in-oil emulsions, oil-water-oil, and water-oil-water. A wide variety of ingredients may be present in either the oil or water phase of the emulsion. That is, the lotion formulation is not particularly limited.

Examples of lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

These same general ingredients may be formulated into a cream rather than a lotion, or into gels, or into solid sticks by utilization of different proportions of the ingredients and/or by inclusion of thickening agents such as gums, carbomers, or other forms of hydrophilic colloids. Very generally, as is known in the art, creams and ointments are typically spreadable in the range from room temperature to skin temperature, and lotions and milks are more flowable within this temperature.

In one or more embodiments, the carrier is water-based. In one or more embodiments, the carrier contains one or more emulsifiers, one or more vegetal waxes, and one or more olive derivatives or extracts.

In one or more embodiments, a surprising enhancement of the skin barrier function is observed when the composition includes a vegetal wax. Advantageously, the vegetal wax promotes a liquid crystal network structure to the emulsion.

In one or more embodiments, a liquid crystal network structure in the compositions of the present invention improves the physiological penetration of active ingredients, increases the barrier integrity, and aids in functional hydration.

Without wishing to be bound by theory, it is believed that carriers including vegetal wax according to the present invention provide a complex combination of fatty acids that are chemically similar to the skin surface lipid composition, and that have the distinctive property to self-emulsify in hydrophilic or lipophilic millieus. In one or more embodiments, the distinctive complex combination of fatty acids represent a unique biomimetic restructuring agent endowed with the double feature of first restoring and maintaining the integrity of the skin barrier and then providing itself the emulsifying base.

Compositions of the present invention containing a liquid crystalline carrier provide liquid crystals with a skin-like fatty acid composition. The highly stable and dermo-compatible liquid crystals are similar to the lipids of the cutaneous barrier. In one or more embodiments, the liquid crystals act as biomimetic restructuring agents and restore the optimal integrity of the skin barrier function and increase the integrity of the stratum corneum barrier function leading to an increased and sustained skin hydration.

The vegetal wax is a vegetable-based liquid crystal promoter that is believed to stabilize oil-in-water emulsions and to improve the texture of emulsions. As a liquid crystal promoter, the vegetal wax re-organizes the emulsion's microscopic structure, acting as an emulsion stabilizing agent. The lamellar liquid crystals produce several bi-layers that enrobe the oil droplets, producing an energy layer preventing coalescence.

In one or more embodiments, the vegetal wax provides the lotion with sebum-control benefits. In one or more embodiments, it provides skin-hydration and a unique texture due to the high water content of the liquid crystalline structure (water incorporated between several bilayers). In one or more embodiments, the vegetal wax influences positively the delivery of active ingredients to the skin.

Examples of vegetal waxes include a blend of cetyl palmitate, sorbitan palmitate and sorbitan olivate such as the blend that is available from B&T S.r.l. under the tradename Oliwax LC.

In one or more embodiments, the composition includes at least one vegetal wax in an amount of at least about 0.5 wt. %, in other embodiments, at least about 0.75 wt. %, in other embodiments, at least about 1 wt. %, in other embodiments, at least about 1.5 wt. %, in other embodiments, at least about 2 wt. %, in other embodiments, at least about 2.5 wt. %, based upon the total weight of the composition.

In one or more embodiments, the composition includes at least one vegetal wax in an amount of up to about 10 weight percent (wt. %), in other embodiments, up to about 8 wt. %, in other embodiments, up to about 5 wt. %, in other embodiments, up to about 3 wt. %, in other embodiments, up to about 2.5 wt. %, in other embodiments, up to about 2 wt. %, in other embodiments, up to about 1.5 wt. %, in other embodiments, up to about 1 wt. %, based upon the total weight of the composition.

In one or more embodiments, the composition includes at least one vegetal wax in an amount of from about 0.5 to about 10 wt. %, in other embodiments, from about 0.75 to about 8 wt. %, and in other embodiments, based upon the total weight of the composition.

In one or more embodiments, the composition further comprises at least one derivative or extract of olives. In one or more embodiments, the olive derivative includes olive oil. In one or more embodiments, the olive derivative includes a cetearylic ester derivative and/or a sorbitan ester derivative. In one or more embodiments, the olive derivative includes a blend of a cetearylic ester derivative and a sorbitan ester derivative. In one or more embodiments, the olive derivative includes a blend having an INCI designation of Cetearyl Olivate (and) Sorbitan Olivate. Cetearyl olivate (and) sorbitan olivate is available from B&T S.r.l. under the tradename OliveM®1000. OliveM is sometimes described as an O/W emulsifier derived from olive oil. It is substantially free of polyethylene oxides (PEG).

In one or more embodiments, it is believed that the cetearylic ester derivative stabilizes the liquid crystals. In certain embodiments, it is believed that the sorbitan ester derivative enhances the emolliency properties of the composition and/or provides easier dispersion for powders. Advantageously, UV filters and pigments may be easily dispersed at high percentages. It is believed that the blend of a cetearylic ester derivative and a sorbitan ester derivative of olive combines a liquid crystal structure with an oleic component derived from olive oil. In one or more embodiments, skin penetration is enhanced, and a soft, silky smooth after-feel is obtained. The substantivity of its composition, being very similar to the human sebum, provides retention of the skin moisture and increases the active ingredient's resistance to water and/or sweat.

It is believed that the blend of a cetearylic ester derivative and a sorbitan ester derivative of olive operates by forming liquid crystals in emulsions, by placing itself at the interface of a two phase system in a preferential direction, placing the polar head into the aqueous phase and the nonpolar tail into the lipidic phase. In one or more embodiments, the postmicellar organization of the blend of a cetearylic ester derivative and a sorbitan ester derivative of olive in water is the typical structure of a liquid crystal reticule, where the bilayer micelles create a multilayer lamellar formation.

In one or more embodiments, emulsions that are formulated with the blend of a cetearylic ester derivative and a sorbitan ester derivative of olive appear very shiny and light and have an original, fresh and silky touch, even if they contain high percentages of lipids. The blend of a cetearylic ester derivative and a sorbitan ester derivative of olive, promoting the formation of this reticular structure inside the emulsion, allows the formulation to contain quite large amounts of natural and polar lipids without affecting the final stability of the emulsion. In one or more embodiments, up to about 25 wt. % of the emulsion may be natural and/or polar lipids.

In one or more embodiments, the composition includes at least one olive derivative or extract in an amount of at least about 0.1 wt. %, in other embodiments, at least about 0.25 wt. %, in other embodiments, at least about 0.5 wt. %, in other embodiments, at least about 0.75 wt. %, in other embodiments, at least about 1 wt. %, in other embodiments, at least about 1.5 wt. %, in other embodiments, at least about 2 wt. %, in other embodiments, at least about 2.5 wt. %, based upon the total weight of the composition.

In one or more embodiments, the composition includes at least one olive derivative or extract in an amount of up to about 20 wt. %, in other embodiments, up to about 18 wt. %, in other embodiments, up to about 15 wt. %, in other embodiments, up to about 10 wt. %, in other embodiments, up to about 8 wt. %, in other embodiments, up to about 5 wt. %, in other embodiments, up to about 3 wt. %, in other embodiments, up to about 2 wt. %, in other embodiments, up to about 1 wt. %, in other embodiments, up to about 0.5 wt. %, based upon the total weight of the composition.

In one or more embodiments, the composition includes at least one olive derivative or extract in an amount of from about 0.1 to about 20 wt. %, in other embodiments, from about 0.5 to about 15 wt. %, based upon the total weight of the composition.

In one or more embodiments, the lotion compositions include olive oil.

In one or more embodiments, the composition includes at least one olive oil in an amount of at least about 0.1 wt. %, in other embodiments, at least about 0.25 wt. %, in other embodiments, at least about 0.5 wt. %, in other embodiments, at least about 0.75 wt. %, in other embodiments, at least about 1 wt. %, in other embodiments, at least about 1.5 wt. %, in other embodiments, at least about 2 wt. %, in other embodiments, at least about 2.5 wt. %, based upon the total weight of the composition.

In one or more embodiments, the composition includes at least one olive oil in an amount of up to about 20 wt. %, in other embodiments, up to about 18 wt. %, in other embodiments, up to about 15 wt. %, in other embodiments, up to about 10 wt. %, in other embodiments, up to about 8 wt. %, in other embodiments, up to about 5 wt. %, in other embodiments, up to about 3 wt. %, in other embodiments, up to about 2 wt. %, in other embodiments, up to about 1 wt. %, in other embodiments, up to about 0.5 wt. %, based upon the total weight of the composition.

In one or more embodiments, the composition includes at least one olive oil in an amount of from about 0.1 to about 20 wt. %, in other embodiments, from about 0.5 to about 15 wt. %, based upon the total weight of the composition.

In one or more embodiments, the carrier includes one or more emulsifiers. Examples of emulsifiers include glycerol esters, in particular glycerol esters of α-hydroxycarboxylic acids and saturated fatty acids. Specific examples include glyceryl stearate. In one or more embodiments, the total amount of the glycerol esters in the composition is advantageously chosen from the range from about 0.1 to about 10.0 wt. %, in one or more embodiments, from about 0.5 to about 6.0 wt. %, based on the total weight of the composition.

In one or more embodiments, the carrier comprises a cold process formulation aid. In one or more embodiments, the carrier comprises at least one wax selected from the group consisting of natural waxes and synthetic waxes and at least one cationic polymer. In one or more embodiments, the carrier comprises one or more C12-C18 fatty acid-C2-C5 polyol esters such as glyceryl monostearate, ethylene glycol monostearate and polyethylene glycol distearate. Examples of polyethylene glycol distearates include PEG-150 distearate. Examples of cationic polymers include polyquaternium polymers, such as polyquaternium-37. The carrier may further comprise one or more fatty alcohols. Examples of fatty alcohols include cetearyl alcohol. Cold process formulation aids are further described in U.S. Patent Application Publication Nos. 2011/0250148, 2011/0250151, 2014/0094558, and 2014/0336308, all of which are incorporated by reference herein.

Compositions or the present invention may further comprise one or more of a wide range of optional ingredients, with the proviso that they do not deleteriously affect the beneficial properties of the composition. The Personal Care Products Council International Cosmetic Ingredient Dictionary and Handbook, Fifteenth Edition 2014, and the 2007 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Examples of optional ingredients, classified by function, include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, humectants, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein include solubilizing agents, sequestrants, and keratolytics, topical active ingredients, deposition enhancers, humectants, moisturizing esters, emulsifying agents, silicone glycols, miscellaneous skin conditioners, thickeners, and/or antimicrobial agents.

Optionally, compositions of the present invention may include one or more pharmacological and/or antibiotic agents, with the proviso that the pharmacological and/or antibiotic ingredient does not deleteriously affect the skin barrier function or skin conditioning properties of the composition. Examples of such agents include, but are not limited to, antifungal agents, antiviral agents, antimicrobial agents, and antiparasitic agents. In one or more embodiments, one or more antimicrobial agents are included. Examples of antimicrobial agents include, but are not limited to, triclosan, also known as 5-chloro-2(2,4-dichlorophenoxy) phenol (PCMX) and available from Ciba-Geigy Corporation under the tradename IRGASAN®; chloroxylenol, also known as 4-chloro-3,5-xylenol, available from Nipa Laboratories, Inc. under the tradenames NIPACIDE® MX or PX; hexetidine, also known as 5-amino-1,3-bis(2-ethylhexyl)-5-methyl-hexahydropyrimidine; chlorhexidine salts including chlorhexidine gluconate and the salts of N,N″-Bis(4-chlorophenyl)-3,12-diimino-2,4,11,14-tetraazatetradecanediimidiamide; 2-bromo-2-nitropropane-1; 3-diol, benzalkonium chloride; cetylpyridinium chloride; alkylbenzyldimethylammonium chlorides; iodine; phenol, bisphenol, diphenyl ether, phenol derivatives, povidone-iodine including polyvinylpyrrolidinone-iodine; parabens; hydantoins and derivatives thereof, including 2,4-imidazolidinedione and derivatives of 2,4-imidazolidinedione as well as dimethylol-5,5-dimethylhydantoin (also known as DMDM hydantoin or glydant); phenoxyethanol; cis isomer of 1-(3-chloroallyl)-3,5,6-triaza-1-azoniaadamantane chloride, also known as quaternium-15 and available from Dow Chemical Company under the tradename DOWCIL™ 200; diazolidinyl urea; benzethonium chloride; methylbenzethonium chloride; glyceryl laurate, transition metal compounds such as silver, copper, magnesium, zinc compounds, hydrogen peroxide, chlorine dioxide, anilides, bisguanidines, a blend of biostatic and fungistatic agents having the INCI name caprylhydroxamic acid (and) propanediol, and mixtures thereof. In one or more embodiments, the composition comprises from about 0.05 to about 3 wt. %, in other embodiments, from about 0.07 to about 2.5 wt. %, in other embodiments, from about 0.09 to about 1 wt. %, in other embodiments, from about 0.1 to about 0.75 wt. %, in other embodiments, from about 0.15 to about 0.5 wt. %, of at least one antimicrobial agents, based upon the total weight of the composition.

In one or more embodiments, the composition may include at least one antibiotic. Examples of antibiotics include asaminoglycoside antibiotics, cephalosporins, carbapenems, quinolone, macrolide antibiotics, penicillins, sulfonamides, tetracyclines, oxazolidinones, lipopeptides, gemifloxacin, ketolides, clindamycin, metronidazole, vancomycin, rifabutin, rifampin, nitrofurantoin, chloramphenicol, erythromycin, gentamicin, vancomycin, ciproflaxin, doxycycline, minocycline, isoniazid, ethambutol, clofazimine, fluoroquinolones, pyrazinamide, streptomycin, ofloxacin, ganciclovir, azithromycin, clarithromycin, dapsone, ampicillin, amphotericin B, ketoconazole, fluconazole, pyrimethamine, sulfadiazine, lincomycin, acyclovir, trifluorouridine, pentamidine, atovaquone, paromomycin, diclazaril, acyclovir, trifluorouridine, foscarnet, sparfloxacin, and pharmaceutically acceptable salts and hydrates thereof.

In one or more embodiments, compositions of the present invention may further include one or more probiotics and/or prebiotics. In one or more embodiments, the one or more probiotics include one or more skin commensal microorganisms which positively affect the skin microbiota. For example, the one or more probiotics can include microorganisms that positively affect the skin surface environment, e.g., by altering the pH or inhibiting growth of pathogenic microorganisms. In one or more embodiments, the one or more probiotics can include one or more microorganisms naturally found on the skin surface of the individual. In one or more embodiments, the one or more probiotics can include one or more microorganism that are not naturally found on the skin surface of the individual, but positively affect the skin surface environment. In one or more embodiments, the one or more probiotics can include one or more engineered microorganisms. For example, the one or more probiotics can include a microorganism genetically engineered to have a property that positively affects the skin surface environment, e.g., by synthesizing and excreting an inhibitor of pathogenic microorganisms. See, e.g., Martin et al. (2013) Microbial Cell Factories, 12:71, which is incorporated herein by reference. In one or more embodiments, the probiotic comprises live probiotic microorganisms. In one or more embodiments, the probiotics may be included in a live form, dead form, semi-active or in deactivated form and fragments or fractions originating from the microorganism either live or dead (e.g., as a lyophilized powder). In one or more embodiments, the probiotic includes culture supernatants of the microorganisms.

In one or more embodiments, the one or more probiotics include one or more bacterial probiotics. See, e.g., U.S. Pat. No. 8,557,560, U.S. Patent Application Publication Nos. 2011/0274676 A1, 2014/0037688 A1, Schrezenmeir & De Vrese (2001) Am. J. Clin. Nutr. 73(suppl):361S-364S, and Gueniche et al. (2009) Exp. Dermatol. 19:e1-e8, all of which are incorporated herein by reference.

In one or more embodiments, the one or more bacterial probiotics include one or more of Firmicutes, Actinobacteria, Bacteriodetes, Proteobacteria, or Cyanobacteria. In one or more embodiments, the one or more bacterial probiotics include one or more of Corynebacteria, Propionibacteria, Micrococci, or Staphylococci. In one or more embodiments, the one or more bacterial probiotics include non-lactic acid and/or lactic acid producing bacteria (LAB) and can include Bacteroides, Bifidobacterium, and Lactobacillus. In one or more embodiments, the one or more bacterial probiotics include certain strains of Aerococcus, E. coli, Bacillus, Enterococcus, Fusobacterium, Lactococcus, Leuconostoc, Melissacoccus, Micrococcus, Oenococcus, Sporolactobacillus, Streptococcus, Staphylococcus, Saccharomyces, Pediococcus, Peptostreptococcus, Proprionebacterium, and Weissella. A wide variety of strains of bacteria are available from the ATCC, Manassas, Va. In one or more embodiments, the one or more probiotics include one or more non-pathogenic strains of pathogenic bacteria.

In one or more embodiments, the one or more probiotic may include a bacterial strain that inhibits a second bacterial strain, e.g., by out competing for resources or by inhibiting the growth of the second bacterial stain. In one or more embodiments, the one or more probiotics include skin commensal microorganism Staphylococcus epidermidis. For example, Staphylococcus epidermidis may be used as a probiotic to modulate growth of pathogenic bacteria on the skin surface by producing microbial peptides that inhibit Staphylococcus aureus biofilm formation and/or by producing lanthionine-containing antibacterial peptides, e.g., bacteriocins, which are known to exhibit antibacterial properties toward certain species of harmful bacteria, e.g., Streptococcus aureus and Streptococcus pyogenes. For example, Staphylococcus epidermidis may be used as a probiotic to stimulate the immune system by influencing the innate immune response of keratinocytes through Toll-like receptor (“TLR”) signaling. For example, Staphylococcus epidermidis may be used as a probiotic to inhibit the action of more virulent microorganisms such as Staphylococcus aureus by occupying receptors on a host cell that also bind the virulent microorganism. See, e.g., Orrice & Segre (2011) Nat. Rev. Microbiol. 9:244-53, which is incorporated herein by reference.

In one or more embodiments, the one or more probiotics can include skin commensal microorganism Propionibacterium acnes. For example, Propionibacterium acnes can be used as a probiotic to consume skin oil and to produce byproducts such as short-chain fatty acids and propionic acid known to help maintain a healthy skin barrier.

In one or more embodiments, the one or more treatment agents include one or more prebiotics. In one or more embodiments, the one or more prebiotics are agents that promote the survival and/or growth of microorganisms of interest on the skin surface of the individual. In one or more embodiments, the one or more prebiotics include at least one of galacto-oligosaccharides, fructo-oligosaccharides, inulin, or lactulose. In one or more embodiments, the one or more prebiotics include one or more of iron, biotin, nicotinic acid, D-pantothenic acid, pyridoxal, pyridoxamine dihydrochloride, thiamin hydrochloride, valine, arginine, galactose, mannose, fructose, sucrose, lactose, or maltose. In one or more embodiments, the one or more prebiotics include one or more of plant derived prebiotics, e.g., derived from acacia gum, konjac, chicory root, Jerusalem artichoke, asparagus, and dandelion greens. See, e.g., U.S. Patent Application Publication NO. 2013/0115317 A1; and Bateni et al. (2013) Am. J. Dermatology Venereology 2:10-14, both of which are incorporated herein by reference.

The composition may further comprise one or more zinc compounds. Examples of zinc compounds include aluminum zinc oxide, ammonium silver zinc aluminum silicate, ethylene/zinc acrylate copolymer, lactobacillus/milk/calcium/phosphorus/magnesium/zinc ferment, lactobacillus/milk/manganese/zinc ferment lysate, luminescent zinc sulfide, magnesium/aluminum/zinc/hydroxide/carbonate, porphyridium/zinc ferment, saccharomyces/zinc ferment, saccharomyces/zinc/iron/germanium/copper/magnesium/silicon ferment, saccharomyces/zinc/magnesium/calcium/germanium/selenium ferment, silicon/titanium/cerium/zinc oxides, sodium zinc cetyl phosphate, sodium zinc histidine dithiooctanamide, zinc acetate, zinc acetylmethionate, zinc adenosine triphosphate, zinc ascorbate, zinc aspartate, zinc borate, zinc borosilicate, zinc carbonate, zinc carbonate hydroxide, zinc cerium oxide, zinc chloride, zinc citrate, zinc coceth sulfate, zinc coco-sulfate, zinc cysteinate, zinc dibutyldithiocarbamate, zinc DNA, zinc formaldehyde sulfoxylate, zinc glucoheptonate, zinc gluconate, zinc glutamate, zinc glycinate, zinc glycyrrhetinate, zinc hexametaphosphate, zinc hydrolyzed collagen, zinc lactate, zinc laurate, zinc magnesium aspartate, zinc myristate, zinc neodecanoate, zinc oxide, zinc palmitate, zinc PCA, zinc pentadecene tricarboxylate, zinc peroxide, zinc phenolsulfonate, zinc picolinate, zinc pyrithione, zinc ricinoleate, zinc rosinate, zinc salicylate, zinc silicates, zinc stearate, zinc sulfate, zinc sulfide, zinc thiosalicylate, zinc undecylenate, zinc undecylenoyl hydrolyzed wheat protein, and zinc zeolite.

It will be understood that recommended amounts of zinc compounds for achieving good barrier properties are about 20 wt. % or higher. Advantageously, as a result of the surprising synergistic effect of the combination of oligopeptides, as well as the liquid crystal carrier, beneficial skin-conditioning and barrier effects are achieved with compositions containing little or no zinc. Therefore, in one or more embodiments, the total amount of zinc compounds may be limited. In one or more embodiments, the total amount of zinc compounds in the composition may be less than about 2 wt. %, in other embodiments, less than about 1 wt. %, in other embodiments, less than about 0.5 wt. %, in other embodiments, less than about 0.1 wt. %, in other embodiments, less than about 0.05 wt. %, based upon the total weight of the composition. In one or more embodiments, the composition is devoid of zinc compounds.

In one or more embodiments, the amount of zinc oxide may be less than about 2 wt. %, in other embodiments, less than about 1 wt. %, in other embodiments, less than about 0.5 wt. %, in other embodiments, less than about 0.1 wt. %, in other embodiments, less than about 0.05 wt. %, based upon the total weight of the composition. In other words, the amount of zinc oxide may be from about zero to about 2 wt. %, based upon the total weight of the composition. In one or more embodiments, the composition is devoid of zinc oxide.

In certain embodiments, the composition comprises one or more humectants. Examples of humectants include propylene glycol, hexylene glycol, 1,4-dihydroxyhexane, 1,2,6-hexanetriol, sorbitol, butylene glycol, propanediols, such as methyl propane diol, dipropylene glycol, triethylene glycol, glycerin (glycerol), polyethylene glycols, ethoxydiglycol, polyethylene sorbitol, glycolic acid, glycolate salts, lactate salts, urea, hydroxyethyl urea, alpha-hydroxy acids, such as lactic acid, sodium pyrrolidone carboxylic acid, hyaluronic acid, chitin, and combinations thereof.

Examples of polyethylene glycol humectants include PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, and PEG-800.

In one or more embodiments, the composition includes at least one humectant in an amount of at least about 0.001 wt. %, in other embodiments, at least about 0.002 wt. %, in other embodiments, at least about 0.005 wt. %, in other embodiments, at least about 0.01 wt. %, in other embodiments, at least about 0.02 wt. %, in other embodiments, at least about 0.05 wt. %, in other embodiments, at least about 0.1 wt. %, in other embodiments, at least about 0.2 wt. %, in other embodiments, at least about 0.5 wt. %, in other embodiments, at least about 0.7 wt. %, in other embodiments, at least about 1 wt. %, in other embodiments, at least about 1.5 wt. %, in other embodiments, at least about 2 wt. %, based upon the total weight of the composition.

In one or more embodiments, the composition includes at least one humectant in an amount of up to about 20 wt. %, in other embodiments, up to about 15 wt. %, in other embodiments, up to about 10 wt. %, in other embodiments, up to about 8 wt. %, in other embodiments, up to about 5 wt. %, in other embodiments, up to about 3 wt. %, based upon the total weight of the composition.

Advantageously, as a result of the surprising synergistic effect of the combination of oligopeptides, as well as the liquid crystal carrier, beneficial skin-conditioning effects are achieved with compositions containing relatively low amounts of humectants. In one or more embodiments, the total amount of humectants in the composition may be less than about 2 wt. %, in other embodiments, less than about 1 wt. %, in other embodiments, less than about 0.5 wt. %, in other embodiments, less than about 0.1 wt. %, in other embodiments, less than about 0.05 wt. %, based upon the total weight of the composition. In one or more embodiments, the composition is devoid of any of the above humectants.

In one or more embodiments, the amount of glycerin may be less than about 2 wt. %, in other embodiments, less than about 1 wt. %, in other embodiments, less than about 0.5 wt. %, in other embodiments, less than about 0.1 wt. %, in other embodiments, less than about 0.05 wt. %, based upon the total weight of the composition. In one or more embodiments, the composition is devoid of glycerin. It is believed that one of the reasons that compositions of the present invention have better aesthetics is due to the lower amount of total raw materials that are required to produce a product with effective skin-conditioning benefits.

In these or other embodiments, the composition comprises one or more conditioning or moisturizing esters that are not olive derivatives. Examples include cetyl myristate, cetyl myristoleate, and other cetyl esters, diisopropyl sebacate, and isopropyl myristate.

In one or more embodiments, the composition includes at least one conditioning or moisturizing ester in an amount of up to about 10% by weight, in other embodiments, up to about 5 wt. %, in other embodiments, up to about 2 wt. %, in other embodiments, up to about 1 wt. %, based upon the total weight of the composition.

In one or more embodiments, the composition includes at least one conditioning or moisturizing ester in an amount of at least about 0.001 wt. %, in other embodiments, at least about 0.002 wt. %, in other embodiments, at least about 0.005 wt. %, in other embodiments, at least about 0.01 wt. %, in other embodiments, at least about 0.02 wt. %, in other embodiments, at least about 0.05 wt. %, in other embodiments, at least about 0.1 wt. %, in other embodiments, at least about 0.2 wt. %, in other embodiments, at least about 0.5 wt. %, in other embodiments, at least about 0.7 wt. %, in other embodiments, at least about 1 wt. %, based upon the total weight of the composition.

In another embodiment each ester that is included is present in an amount of from about 0.5 to about 5% by weight, in another embodiment from about 1 to about 2% by weight, based upon the total weight of the composition.

On the other hand, as a result of the surprising synergistic effect of the combination of oligopeptides, as well as the liquid crystal carrier that includes a vetegetal wax and an olive derivative or extract, additional moisturizing esters are not required, or may be present in relatively low amounts. In one or more embodiments, the total amount of moisturizing esters that are not olive-derived in the composition may be less than about 1 wt. %, in other embodiments, less than about 0.5 wt. %, in other embodiments, less than about 0.1 wt. %, in other embodiments, less than about 0.05 wt. %, based upon the total weight of the composition. In one or more embodiments, the composition is devoid of moisturizing esters that are not olive-derived.

In any of the embodiments described above, the composition may be a gel, cream, lotion, ointment, and the like, and may also contain one or more thickening agents. Examples of thickeners include stearyl alcohol, cationic hydroxy ethyl cellulose (Ucare; JR30), hydroxy propyl methyl cellulose, hydroxy propyl cellulose (Klucel), chitosan pyrrolidone carboxylate (Kytamer), behenyl alcohol, zinc stearate, and emulsifying waxes, including but not limited to Incroquat and Polawax. Other thickening and/or gelling agents suitable for incorporation into the anti-irritant gels, creams, lotions or ointments described herein include, for example, an addition polymer of acrylic acid, a resin such as Carbopol® ETD 2020, guar gum, acacia, acrylates/steareth-20 methacrylate copolymer, agar, algin, alginic acid, ammonium acrylate copolymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium tri silicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof.

Examples of thickeners/rheology modifiers include associative polymers. Associative polymers include non-ionic polymeric thickeners. In one or more embodiments, the associative polymer includes a hydrophilic backbone and hydrophobic end groups. In one or more embodiments, the non-ionic polymer includes urethane-based and polyether polyol-based associative thickeners.

Typical amounts of the above thickeners are from about 0.6 to about 2 wt. %, based upon the total weight of the composition.

In one or more embodiments, the composition may be thickened with polyacrylate thickeners such as those conventionally available and/or known in the art. Examples of polyacrylate thickeners include carbomers, acrylates/C10-30 alkyl acrylate crosspolymers, copolymers of acrylic acid and alkyl (C5-C10) acrylate, copolymers of acrylic acid and maleic anhydride, and mixtures thereof. Polyacrylate thickeners are further described in U.S. Patent Application Publication No. 2010/0317743 A1, which is hereby incorporated by reference.

In one or more embodiments, strong acids and other ingredients that may attack the peptide bonds in the oligopeptide may be limited. In one or more embodiments, the amount of protein denaturants is limited. In one or more embodiments, elevated temperatures are avoided.

Advantageously, ingredients that are typically required for barrier and/or hydration products may be limited, or eliminated altogether, which may lead to improved rheology, aesthetics, and/or stability. In one or more embodiments, the compositions according to the present invention include from zero up to about 2 wt. % dimethicone. In one or more embodiments, the total amount of dimethicones in the composition is less than about 2 wt. %, in other embodiments, less than about 1.5 wt. %, in other embodiments, less than about 1 wt. %, in other embodiments, less than about 0.5 wt. %, in other embodiments, less than about 0.1 wt. %, in other embodiments, less than about 0.05 wt. %, based upon the total weight of the composition. In one or more embodiments, the composition is devoid of dimethicones.

In one or more embodiments, the amount of petrolatum may be limited. More specifically, in one or more embodiments, the total amount of petrolatum in the composition may be less than about 2 wt. %, in other embodiments, less than about 1 wt. %, in other embodiments, less than about 0.5 wt. %, in other embodiments, less than about 0.1 wt. %, in other embodiments, less than about 0.05 wt. %, based upon the total weight of the composition. In one or more embodiments, the composition is devoid of petrolatum.

In one or more embodiments, the amount of mineral oil may be limited. More specifically, in one or more embodiments, the total amount of mineral oil in the composition may be less than about 2 wt. %, in other embodiments, less than about 1 wt. %, in other embodiments, less than about 0.5 wt. %, in other embodiments, less than about 0.1 wt. %, in other embodiments, less than about 0.05 wt. %, based upon the total weight of the composition. In one or more embodiments, the composition is devoid of mineral oil.

It is also contemplated that ingredients identified throughout this specification, including but not limited to the oligopeptides, may be individually or combinatorially encapsulated for delivery to a target area such as skin. Non-limiting examples of encapsulation techniques include the use of liposomes, vesicles, and/or nanoparticles (e.g., biodegradable and non-biodegradable colloidal particles comprising polymeric materials in which the ingredient is trapped, encapsulated, and/or absorbed—examples include nanospheres and nanocapsules) that can be used as delivery vehicles to deliver the ingredient to skin. Encapsulation is further described in U.S. Pat. Nos. 6,387,398, 6,203,802, and 5,411,744, all of which are incorporated by reference herein. Encapsulation of the oligopeptides may allow the use of ingredients that would otherwise be limited, such as strong acids and ethanol.

The composition may be prepared by simply mixing the components together. The order of addition is not particularly limited, but may advantageously be selected based upon the solubility of the various ingredients.

In one or more embodiments, the composition may be prepared by combining at elevated temperature water, at least one emulsifier, a vegetal wax, and one or more olive derivatives or extracts, and mixing until a homogeneous mixture is obtained. In one or more embodiments, the elevated temperature is about 82° C.

After the mixture has cooled to about 52° C. or less, an additional olive derivative or extract may be added, along with other optional ingredients, if desired.

After the mixture has cooled to about 38° C. or less, the oligomeric peptides may be added, along with other optional ingredients such as preservatives, if desired.

Mixing is continued until a homogenous mixture is obtained.

In one or more embodiments, the compositions of the present invention are smooth and uniform, and easy to spread, leaving a uniform residue on skin. In one or more embodiments, the compositions are stable and pass standard laboratory stability test methods. In one or more embodiments, compositions were stable at 4, 25, 40 and 50° C. for at least three months. In one or more embodiments, compositions were also stable under freeze-thaw test conditions.

Sensory aesthetics deals with evaluating cosmetic preparations on the basis of sensory impressions. A sensory assessment of a cosmetic is made by reference to visual, olfactory and haptic impressions. Haptic impressions include sensations of the sense of touch, which relate primarily to structure and consistency of the product, i.e., texture. Texture is an aesthetic property of cosmetic products that is very important for the consumer, but which can only be quantitatively measured with difficulty. Texture is generally understood to mean those properties of a cosmetic which relate to the structure of the preparation, and are perceived by the sense of touch.

A method of sensory analysis that is often used in research and development is the difference test. A sample is compared to a control sample, and differences are perceived. The use of groups of trained test persons, screening the testers from one another, and statistical evaluation of the data all help to counter the inherent subjectivity of the sensory analysis. Advantageously, this product has a light and easy to spread sensory experience that is desired by end users. Also leaves a light film on skin that is perceived as continued protection.

Compositions of the present invention may also be characterized by reference to viscosity and/or rheological properties. In one or more embodiments, the viscosity may be expressed as a standard, single-point type viscosity, as measured on a Brookfield Digital viscometer at a temperature of about 20° C., using spindle T-D, heliopath, at a speed of 10 rpm. In one or more embodiments, the compositions may have a viscosity of from about 2000 to about 120,000 cPs.

In one or more embodiments, compositions of the present invention may be characterized as lotions, having a viscosity of less than about 120,000 centipoise (cPs), in other embodiments, less than about 100,000, and in other embodiments, less than about 75,000 cPs. In one or more embodiments, the lotion compositions may have a viscosity of from about 3000 to about 50,000 cPs, in other embodiments, from about 4000 to about 30,000 cPs.

In one or more embodiments, compositions of the present invention may be characterized as serum, having a viscosity of from about 2000 to about 3000 cPs.

In one or more embodiments, compositions of the present invention may be characterized as creams, having a viscosity of from about 30,000 to about 100,000 cPs, in other embodiments from about 50,000 to about 80,000 cPs.

In one or more embodiments, compositions according to the present invention are pourable at room temperature, i.e. a temperature in the range of from about 20 to about 25° C. In one or more embodiments, the lotion formulations are viscous enough to hold a shape or not flow for a desired period of time. In other embodiments, compositions of the present invention are creams or ointments, and are not pourable and do not flow at room temperature and will not conform to a container when placed into the container at room temperature.

The term yield stress point is understood as meaning the smallest shear stress above which a plastic material behaves in rheological terms like a liquid. The yield stress point may be used to indicate the amount of shear stress that is needed to initiate flow, relating to the ability to both pump and spread the product. The yield stress point may be determined by plotting a flow curve.

Flow curves of the compositions according to the invention may be prepared using an SR-2000 from Rheometric Scientific (now TA-Instruments), StressTec High Resolution Research Rheometer, or the like. This instrument is a shear-stress-controlled rheometer with an air-bearing transducer. The measurement system consists of a parallel plate measurement system (so-called plate/plate arrangement) where the lower plate can be temperature controlled. The measurement is carried out at a measuring temperature of 25° C. In one or more embodiments, the measurement method includes a linear shear stress-time slope with a strain rate of 40 Pa/min starting at 0 Pa.

Advantageously, compositions of the present invention become less viscous when relatively low amount of stress is applied. Thus, the compositions are easily spreadable. In one or more embodiments, compositions of the present invention may be characterized in that the flow curve indicates a significant drop in viscosity at a stress of about 10 pascals (Pa), in other embodiments, at a stress of about 50 Pa, in other embodiments, at a stress of about 75 Pa, and in other embodiments, at a stress of about 100 Pa. By significant drop is meant a rather sudden drop, as opposed to a gradual decline.

In one or more embodiments, the yield stress point is less than about 1000 Pa, at 1 s⁻¹ at about 20° C., in other embodiments, the yield stress point is less than about 500 Pa, in other embodiments, less than about 100 Pa, in other embodiments, less than about 50 Pa, at 1 s⁻¹ at about 20° C.

In one or more embodiments, the compositions of the present invention are thixotropic. Another way to express this feature is to say that the compositions exhibit a thixotropic rheology. Thixotropy is a time-dependent shear thinning property. In general, the viscosity of thixotropic compositions decreases when shear forces are applied. Certain gels or fluids that are viscous under static conditions will flow, i.e. become less viscous, over time when stress is applied. The compositions then take a fixed amount of time to return to a more viscous state.

Advantageously, compositions of the present invention recover quickly when the stress is removed. In one or more embodiments, the shear-thinning effect is substantially reversible. Thus, in one or more embodiments, compositions of the present invention exhibit a first viscosity under static conditions, a second, reduced viscosity when shear force is applied, and then, when the shear force is removed, the viscosity of the composition returns to the first viscosity, or to a viscosity that is substantially the same as the first viscosity.

In one or more embodiments, the second viscosity is at least 25% of the first viscosity, in other embodiments, the second viscosity is at least 50% of the first viscosity, in other embodiments, at least 75% of the first viscosity, when a stress of up to about 10 to about 100 Pa is applied as described above. In one or more embodiments, the second viscosity is at least 85% of the first viscosity, in other embodiments, the second viscosity is at least 90% of the first viscosity, in other embodiments, at least 95% of the first viscosity, in other embodiments, at least 98% of the first viscosity, when a stress of up to about 10 to about 100 Pa is applied as described above. Stated another way, the reduction in viscosity in relation to stress applied at a stress of about 100 Pa is less than about 75%, in other embodiments, less than about 50%, in other embodiments, less than about 25%, based upon the original viscosity of the composition. In one or more embodiments, the reduction in viscosity in relation to stress applied at a stress of about 10 to about 100 Pa is less than about 20%, in other embodiments, less than about 10%, in other embodiments, less than about 5%, based upon the original viscosity of the composition.

In one or more embodiments, the composition is topically applied to skin. In one or more embodiments, the composition may be topically applied to an affected skin area in a predetermined or as-needed regimen. In one or more embodiments, the composition is included as part of a skin cleansing or sanitizing regimen.

Advantageously, in one or more embodiments, lotions according to the present invention provide protection against irritants. Irritants may include chemical irritants, biological irritants, such as result from incontinence. In one or more embodiments, lotions according to the present invention enhance the healing of damaged skin. Damaged skin may be the result of environmental factors, aging, or disease. In one or more embodiments, lotions according to the present invention prevent pressure ulcers.

Thus, the present invention further provides a method for reducing the irritancy potential of a skin cleanser or sanitizer. The method includes the step of combining a skin cleanser or sanitizer composition with one or more oligopeptides prior to form a less irritating skin cleanser or sanitizer composition. The method includes the further step of contacting the skin with the less irritating composition for a period sufficient to cleanse and/or sanitize the skin. In one or more embodiments, when the amount of skin irritation is measured, as for example by testing the IL-8 secretion, the amount of skin irritation is reduced, compared to when the method is repeated but using the same skin cleanser or sanitizer composition without any oligopeptide. In one or more embodiments, compositions containing two or more oligopeptides provide a synergistic reduction of the skin irritation potential of the compositions.

Advantageously, in one or more embodiments, skin cleansers and sanitizers containing one or more oligopeptide according to the present invention enhance the skin barrier function, when compared to the same skin cleanser or sanitizer but not containing one or more oligopeptide according to the present invention.

Thus, the present invention further provides a method for the treatment of the skin comprising the step of contacting the skin with a cosmetically or pharmaceutically acceptable amount of the compositions described above. Advantageously, the skin condition is improved after contact with the composition. In one or more embodiments, when the skin barrier function is assessed, as for example by testing the skin for cell adhesion proteins, the amount of skin barrier function is improved, compared to when the method is repeated but using the same composition without the two or more oligopeptides. In one or more embodiments, compositions containing two or more oligopeptides, a vegetal wax and a derivative or extract of olives provide a synergistic enhancement of the skin barrier function.

Advantageously, compositions and methods of the present invention may be useful to treat a variety of skin conditions that result in inflammation or erythema. For example, inflammation or erythema can result from external causes such as sun or wind burn or irritating soaps or cleansers. It is also known that inflammation and erythema can be caused from inherent conditions such as rosacea, atopic dermatitis, or allergic skin reactions.

In one or more embodiments, the compositions of the present invention may be formulated as a spray cleansing lotion. General characteristics of spray products are further described in U.S. Patent Application Publication No. 2010/0239624 A1, which is incorporated by reference herein. Advantageously, compositions of the present invention are well suited for spray applications, because they are relatively thin compared to compositions containing zinc compounds. For the same reason, compositions of the present invention are also useful in glide gel and roll on products.

In one or more embodiments, the invention further provides wipes or other fibrous structures comprising the compositions as described herein. Suitable wipes and fibrous structures are described, for example, in U.S. Patent Application Publication Nos. 2010/0239624 A1 and 2013/0004602 A1, both of which are incorporated by reference herein. In one or more embodiments, compositions of the present invention are imbedded in diapers, wipes, tissues, and/or bandages.

In one or more embodiments, the compositions of the present invention may be employed to cover and protect skin wounds or ulcers, such as pressure ulcers. Advantageously, the skin cover is easily applied, does not need to be removed, protects the skin from irritants and contaminants, and similarly, irritants cannot get trapped under the cover.

The present invention provides a method of cleaning and treating decubitus ulcers. A patient in need of treatment of one or more decubitus ulcers is identified. The compositions of the present invention are applied to the skin area containing the decubitus ulcer. Advantageously, the composition provides cleaning to the one or more decubitus ulcers, provides wound healing, and provides a moisture barrier. In one or more embodiments, compositions according to the present invention prevent infection and accelerate healing.

In general, compositions may be assessed for sensory aesthetic characteristics by an expert sensory panel, for example as described in U.S. Patent Application Publication No. 2006/0159649 A1, which is hereby incorporated by reference. The '649 publication also describes methods for assessing rheological characteristics, TEWL, in vitro skin retention test, controlled application dryness test with wash protocols, and skin hydration (corneometer).

Compositions of the present invention have many advantages, including the following. Because the compositions do not have a base odor like petrolatum-based products, compositions may be fragrance-free, or may more easily be formulated with a pleasing fragrance. Rheological properties allow for ease of application with minimum contact and minimum skin tearing. The synergistic improvement in skin hydration and barrier repair make the compositions useful for dry skin and dry lip repair, soothing, healing skin cracking, acne, UV damage, aging, cuts and burns. The compositions provide a barrier against pollutants, irritants, infection, blisters, pressure ulcers, chaffing, diaper rash, tough soils.

In order to demonstrate the practice of the present invention, the following examples have been prepared and tested. The examples should not, however, be viewed as limiting the scope of the invention. The claims will serve to define the invention.

### EXAMPLES

### Testing Methods—Part 1

IL-*ELISA

Interleukin 8 (IL-8) is a chemokine and proinflammatory cytokine produced by macrophages and other cell types such as epithelial cells. It is secreted from keratinocytes in skin in response to inflammatory stimuli. IL-8 is secreted and is an important mediator of the immune reaction in the innate immune system response. IL-8 overexpressed is a biomarker of skin irritation.

For Control A, human dermal keratinocytes are left untreated. No irritation is expected, and therefore Control A provides a baseline. For Control B, IL-8 is induced in human dermal keratinocytes by applying a surfactant mixture that is a combination of sodium laureth sulfate and polyquaternium-10. For all other samples, the human dermal keratinocytes are co-treated with the surfactant mixture and a composition containing the ingredient of interest. Decreased Il-8 expression reflects the ingredient's anti-irritation activity.

In order to carry out the test method, an assay kit was employed that was obtained from R&D Systems: Human CXCL8/IL-8 Quantikine ELISA Kit.

The following steps were followed: 1. Bring all reagents and samples to room temperature before use. 2. Prepare all reagents, standard dilutions, and samples. 3. Remove excess microplate strips from the plate frame, return them to the foil pouch containing the desiccant pack, and reseal. 4. Add 100 μL of Assay Diluent to each well. 5. Add 50 μL of Standard, control, or sample to each well. Cover with a plate sealer, and incubate at room temperature for 2 hours. 6. Aspirate each well and wash, repeating the process 3 times for a total of 4 washes. 7. Add 100 μL of Conjugate to each well. Cover with a new plate sealer, and incubate at room temperature for 1 hour. 8. Aspirate and wash 4 times. 9. Add 200 μL Substrate Solution to each well. Incubate at room temperature for 30 minutes, making sure to protect the wells from the light. 10. Add 50 μL of Stop Solution to each well. The liquid was removed from the well and, using a colorimeter, absorbance was measured at 450 nanometers (nm) within 30 minutes. Wavelength correction was set to 540 nm or 570 nm.

MTT Assay

The MTT assay is a colorimetric assay for assessing cell viability, cell proliferation, and/or cytotoxicity. NAD(P)H-dependent cellular oxidoreductase enzymes may, under defined conditions, reflect the number of viable cells present. These enzymes are capable of reducing the tetrazolium dye MTT 3-(4,5-dimethylthiazol-2-yl)-2,5-diphenyltetrazolium bromide to insoluble formazan, which has a purple color. MTT assay can also be used to measure cytotoxicity (loss of viable cells) or cytostatic activity (shift from proliferative to resting status) of potential medicinal agents and toxic materials.

Controls A and B described above for the IL-8 Assay were also employed in this test. The mitigating effect of the test samples on the effect of Control B on the keratinocytes was measured. More specifically, while Control B has a negative effect on cell viability, cell proliferation, and/or cytotoxicity, this mitigation of this negative effect was determined by measuring the reduction of MTT.

The following steps were followed; Once the liquid was removed from the wells for the IL-8 Assay described above, 100 μl/well of 0.5 mg/ml of MTT in phenol red-free DMEM (cell culture medium) was added into each of the 96-well plates. After incubating 1 hour at 37° C., all liquid was removed (MTT solution) from the wells of the culture plate. Then 100 μl of DMSO was added to each well to completely dissolve the purple product. Absorption was measured using a plate reader at 550 nm wavelength.

Cell-Cell Junction

Tight Junctions are the closely associated areas of two cells whose membranes join together forming a virtually impermeable barrier to fluid. A desmosome is a cell structure specialized for cell-to-cell adhesion. A type of junction complex, they are localized spot-like adhesions randomly arranged on the lateral sides of plasma membranes. Desmosomes are molecular complexes of cell adhesion proteins and linking proteins that attach the cell surface adhesion proteins to intracellular keratin cytoskeletal filaments.

The cell adhesion proteins of the desmosome, desmoglein (DSG) and desmocollin (DSC), are members of the cadherin family of cell adhesion molecules. They are biomarker of skin tight junctions. In particular, DSG1 is a biomarker for cell binding, the higher the expression, the better skin cell-cell junction and the better skin barrier function will be. DSC3 is a protein in humans that is encoded by the DSC3 gene, the higher the expression, the better skin cell-cell junction and the better skin barrier function will be.

In the present method, keratinocytes were treated with the sample compositions in a 6-well plate overnight. After washing with cold phosphate-buffered saline (PBS), total RNAs were prepared from each well. Real-Time Quantitative Reverse Transcription PCR (qRT-PCR) was performed to detect the target genes (DSC1 and DSG3) expression level using a One-step TaqMan® RT-PCR kit (Life Technologies).

### Test Results

Aqueous solutions of acetyl hexapeptide-46 and acetyl hexapeptide-38 were prepared by dilution to achieve the concentrations shown in Table 1. Acetyl hexapeptide-46 was obtained from Lipotec under the tradename Delisens.™ Delisens™ is a proprietary blend of butylene glycol, water, citric acid and acetyl hexapeptide-46, containing 0.025 wt. % acetyl hexapeptide-46. Acetyl hexapeptide-38 was obtained from Lipotec under the tradename Adifyline™. Adifyline™ is a proprietary blend of butylene glycol, water and acetyl hexapeptide-38, containing 0.05 wt. % acetyl hexapeptide-38.

The concentration of acetyl hexapeptide shown in the following table represents the concentration of the active ingredient. Thus, for example, in preparing Example 1A, 2 grams of Adifyline® was mixed with 98 g deionized water to prepare a solution that was 2 wt % Adifyline and 10 parts per million by weight (ppm) acetyl hexapeptide-38, based upon the total weight of the solution.

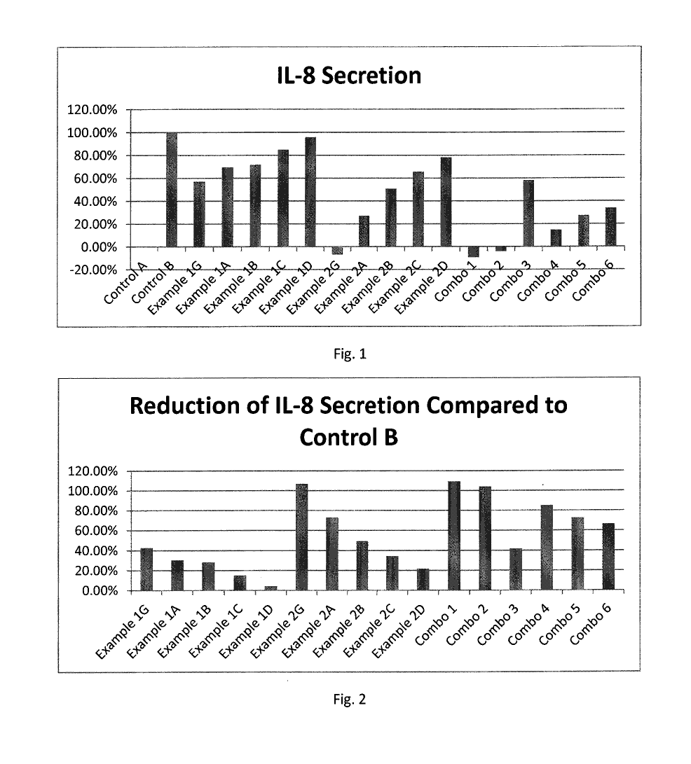

The samples were tested for IL-8 secretion as described in the test method above. That is, for Control A, human dermal keratinocytes were left untreated. For Control B, IL-8 a surfactant mixture that was a combination of sodium laureth sulfate and polyquaternium-10. For all other samples, the human dermal keratinocytes were co-treated with the surfactant mixture and a composition containing the ingredient of interest. Decreased 11-8 expression reflects the ingredient's anti-irritation activity. The results are summarized in Table 2 and shown graphically in FIG. 1. As can be seen in FIG. 1, both acetyl hexapeptide-38 and acetyl hexapeptide-46 reduce the IL-8 secretion, when compared to Control B.

The amount of the reduction in IL-8 secretion for the test samples (subtracting from 100% for Control B) is summarized in Table 2 and shown graphically in FIG. 2. It can be seen that the combination of acetyl hexapeptide-46 and acetyl hexapeptide-38 produce an enhanced reduction in IL-8 secretion, particularly for the higher concentrations.

TABLE 1

| | | Acetyl | | Acetyl |
|---|---|---|---|---|
| | Adifyline | Hexapeptide-38 | Delisens | Hexapeptide-46 |
| Example | Wt. % | ppm | Wt. % | ppm |
| 1G | 3 | 15 | — | — |
| 1A | 2 | 10 | — | — |
| 1B | 1 | 5 | — | — |
| 1C | 0.5 | 2.5 | — | — |
| 1D | 0.2 | 1 | — | — |
| 2G | — | — | 3 | 7.5 |
| 2A | — | — | 2 | 5 |
| 2B | — | — | 1 | 2.5 |
| 2C | — | — | 0.5 | 1.25 |
| 2D | — | — | 0.2 | 0.5 |
| Combo 1 | 3 | 15 | 3 | 7.5 |
| Combo 2 | 2 | 10 | 2 | 5 |
| Combo 3 | 1 | 5 | 1 | 2.5 |
| Combo 4 | 1 | 5 | 2 | 5 |
| Combo 5 | 0.5 | 2.5 | 2 | 5 |
| Combo 6 | 0.2 | 1 | 2 | 5 |

TABLE 2

| | | Reduction of | |
|---|---|---|---|
| | | IL-8 Secretion | |
| | | Compared to | Standard |
| Example | IL-8 secreted | Control B | Deviation |
| Control A | 0.00% | — | 2.00% |
| Control B | 100% | — | 16.70% |
| 1G | 57.20% | 42.80% | 4.40% |
| 1A | 69.60% | 30.40% | 18.60% |
| 1B | 71.70% | 28.30% | 6.60% |
| 1C | 84.90% | 15.10% | 9.60% |
| 1D | 95.80% | 4.20% | 11.90% |
| 2G | −6.90% | 106.90% | 1.10% |
| 2A | 27.10% | 72.90% | 2.70% |
| 2B | 50.90% | 49.10% | 4.60% |
| 2C | 65.60% | 34.40% | 5.00% |
| 2D | 78.20% | 21.80% | 4.00% |
| Combo 1 | −9.40% | 109.40% | 1.10% |
| Combo 2 | −4.10% | 104.10% | 1.10% |
| Combo 3 | 58.30% | 41.70% | 6.10% |
| Combo 4 | 14.90% | 85.10% | 0.40% |
| Combo 5 | 27.40% | 72.60% | 3.60% |
| Combo 6 | 33.70% | 66.30% | 2.60% |

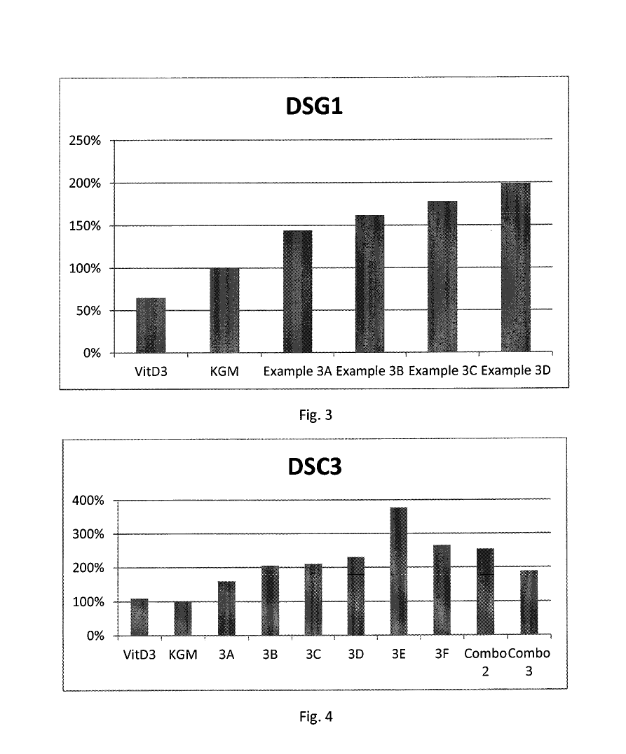

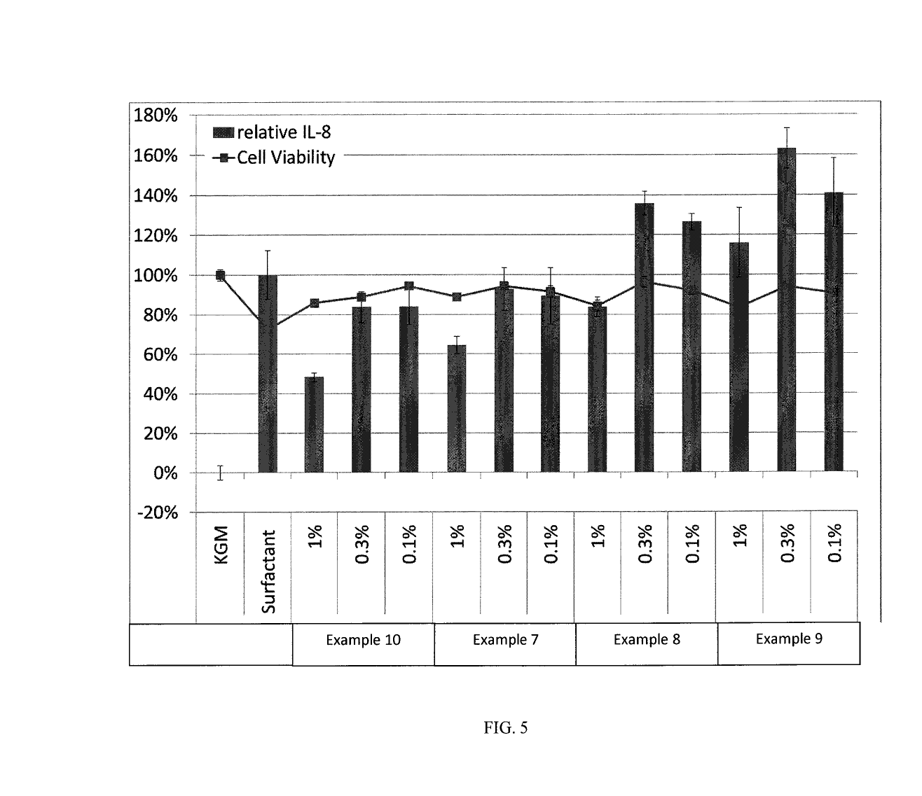

Samples containing various amounts of acetyl hexapeptide-38 and/or acetyl hexapeptide-46 were tested for cell-cell junction, as described above. Solution of Vitamin D3 and keratinocyte growth medium (KGM) were used for comparison. The results are summarized in Tables 3-4 below, and graphically represented in FIGS. 3-4. It can be seen that compositions containing acetyl hexapeptide-38 and/or acetyl hexapeptide-46 enhanced cell-cell junction when compared to Vitamin D3 and KGM.

TABLE 3

| Example | Composition | Conc. (ppm) | DSC3 % |
|---|---|---|---|
| | VitD3 | | 109 |
| | KGM | | 100 |
| 3A | Acetyl hexapeptide-38 | 15 | 160 |
| 3B | Acetyl hexapeptide-38 | 7.5 | 205 |
| 3C | Acetyl hexapeptide-38 | 5 | 210 |
| 3D | Acetyl hexapeptide-46 | 7.5 | 230 |
| 3E | Acetyl hexapeptide-46 | 5 | 377 |
| 3F | Acetyl hexapeptide-46 | 2.5 | 265 |
| Combo 2 | Acetyl hexapeptide-38 + -46 | 5 + 2.5 | 254 |
| Combo 3 | Acetyl hexapeptide-38 + -46 | 10 + 5 | 188 |

TABLE 4

| Example | Composition | Conc. (ppm) | DSG1 % |
|---|---|---|---|
| | VitD3 | | 65 |
| | KGM | | 100 |
| 3B | Acetyl hexapeptide-38 | 7.5 | 144 |
| 3C | Acetyl hexapeptide-38 | 5 | 178 |
| 3E | Acetyl hexapeptide-46 | 5 | 162 |
| 3F | Acetyl hexapeptide-46 | 2.5 | 199 |

Samples were prepared and tested as described above, except that pentapeptides were employed instead of hexapeptides. The concentrations of the samples tested are shown below, as well as the test results. The concentration refers to the parts per million by weight of active in the sample tested.

TABLE 5

| | | | Reduction of | |
|---|---|---|---|---|
| | Concentration | | IL-8 Secretion | |
| | Pentapeptide | IL-8 | Compared to | |
| Example | (ppm) | secreted | Control B | MTT |
| Control A | — | 0.00% | — | 100 |
| Control B | — | 100% | — | 57 |
| 4A | 1 | 67 | 33 | 80 |
| 4B | 5 | 59 | 41 | 81 |
| 4C | 10 | 58 | 42 | 82 |
| 4D | 15 | 64 | 36 | 78 |
| 4E | 20 | 85 | 15 | 75 |
| 5A | 0.2 | 47 | 53 | 81 |
| 5B | 0 | 10 | 90 | 54 |
| 5C | 2 | −3 | 100+ | 37 |
| 5D | 2.5 | −6 | 100+ | 32 |
| 5E | 3 | 0 | 100 | 21 |
| 5F | 3.5 | 21 | 79 | 12 |
| 6A | 5 | 6 | 94 | 97 |
| 6B | 15 | 5 | 95 | 94 |
| 6C | 25 | 9 | 91 | 65 |
| 6D | 35 | 6 | 94 | 44 |
| 6E | 40 | 3 | 97 | 32 |

It can be seen that compositions containing the tested pentapeptides produce an enhanced reduction in IL-8 secretion, and that they produce an enhanced cell-cell junction when compared to Vitamin D3 and KGM.

### Testing Methods—Part 2

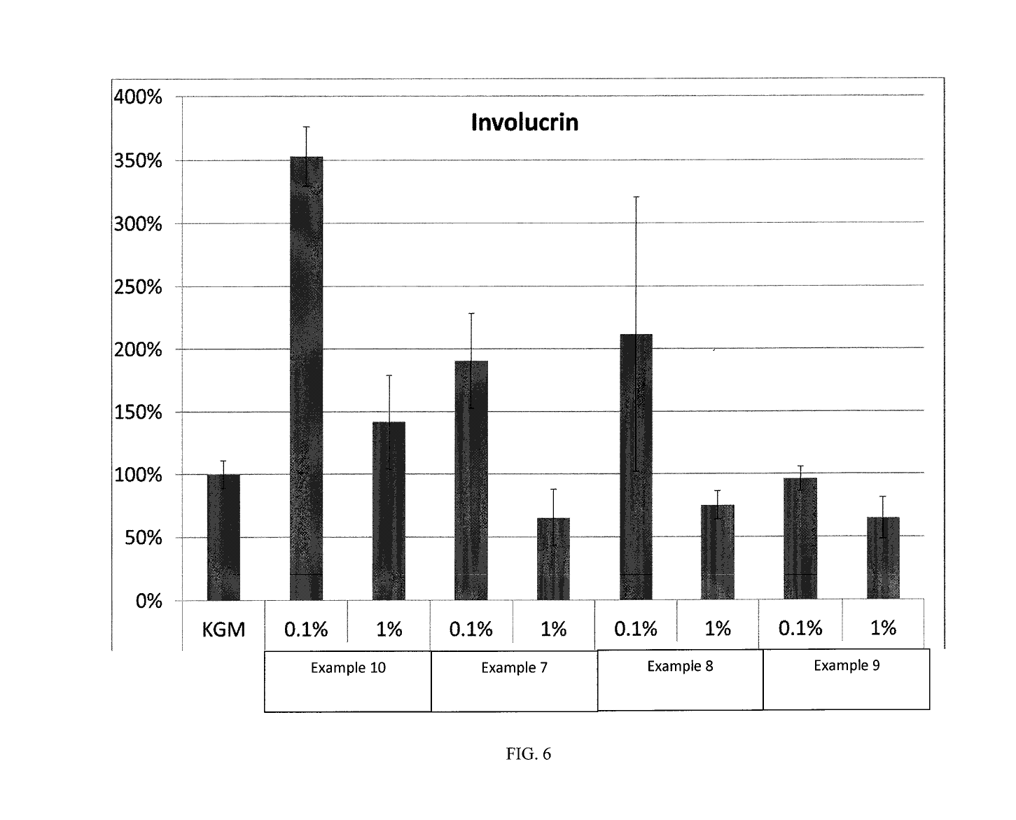

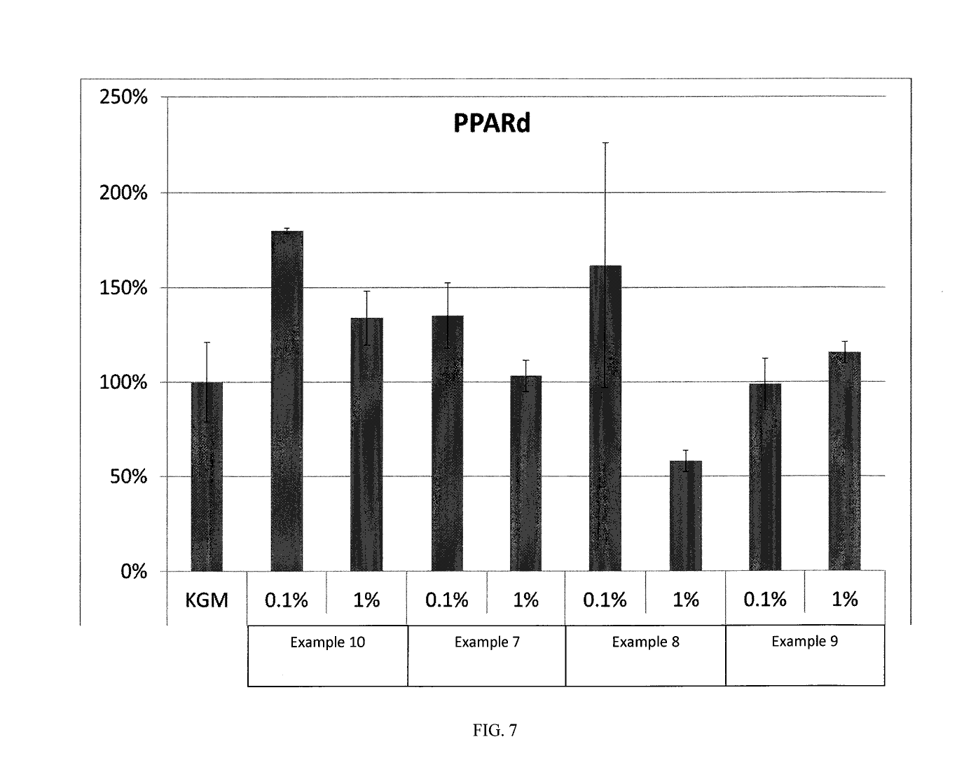

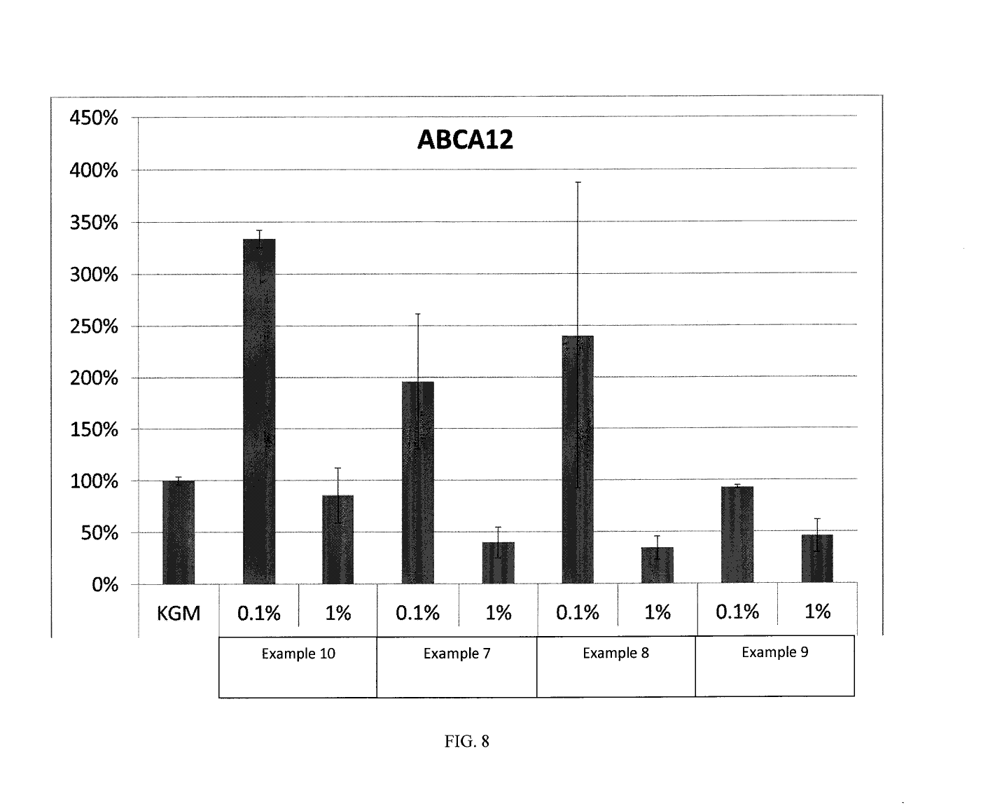

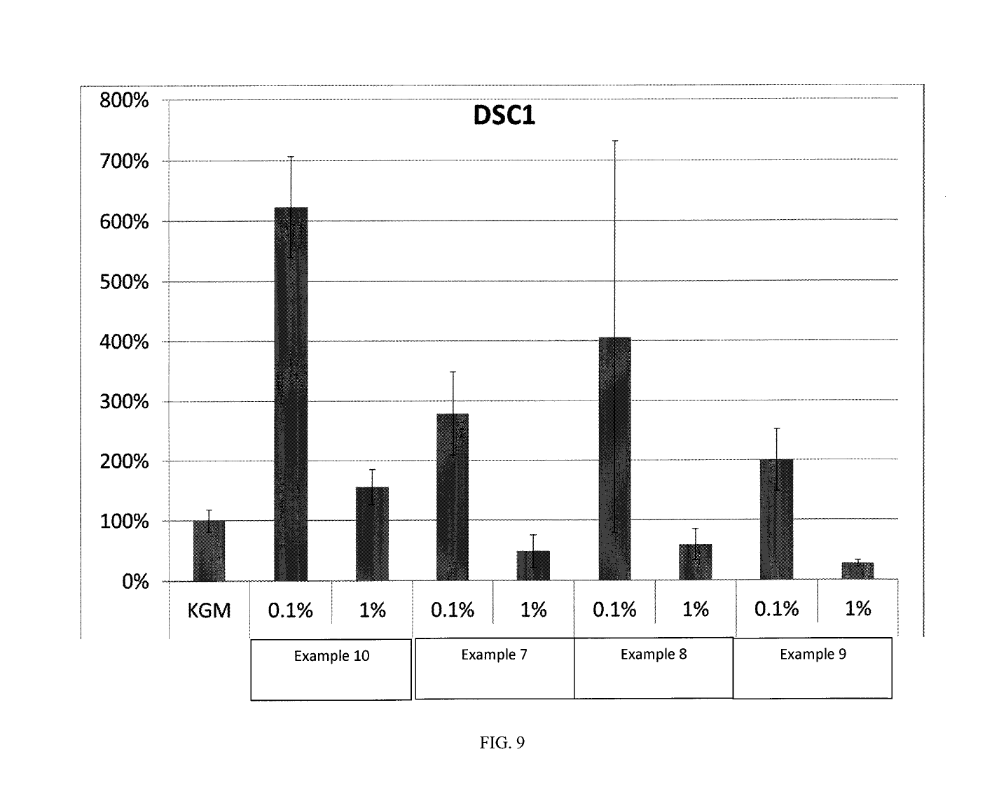

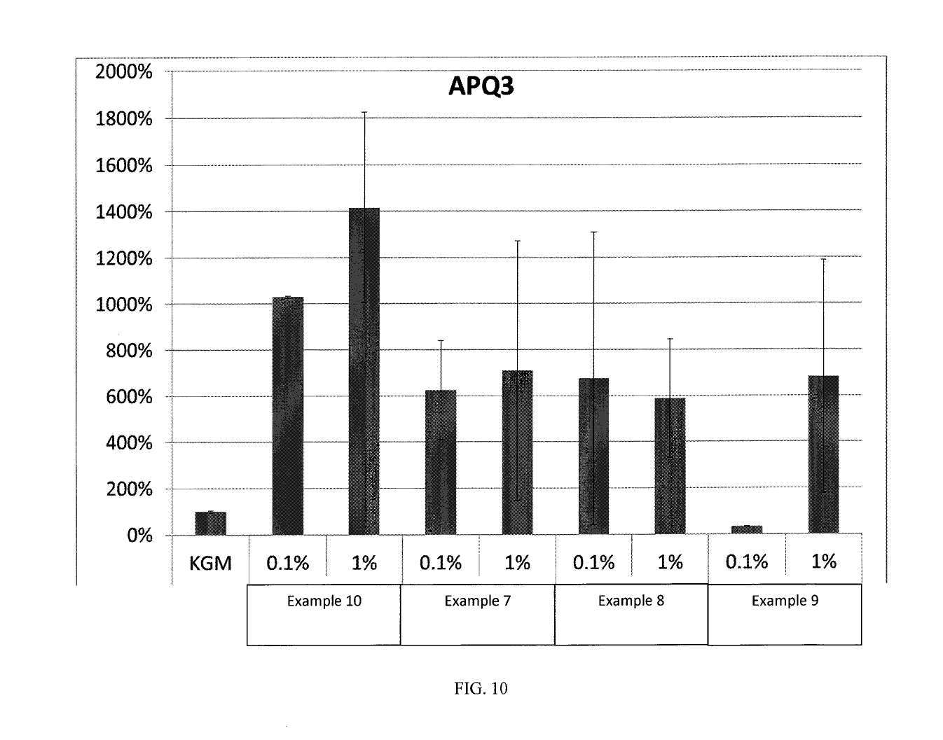

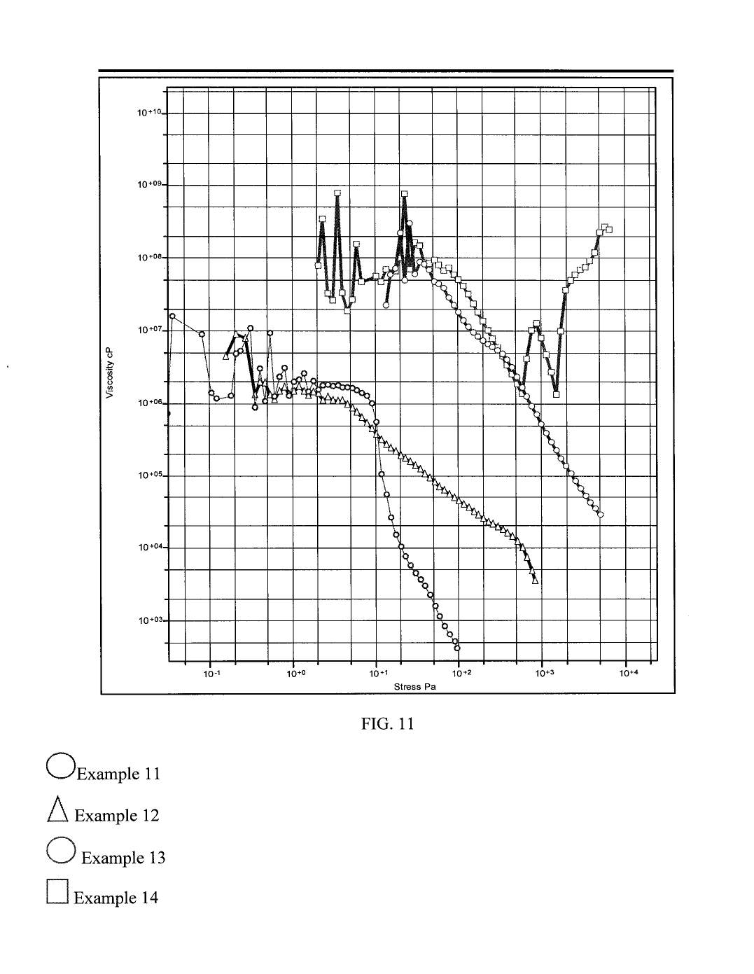

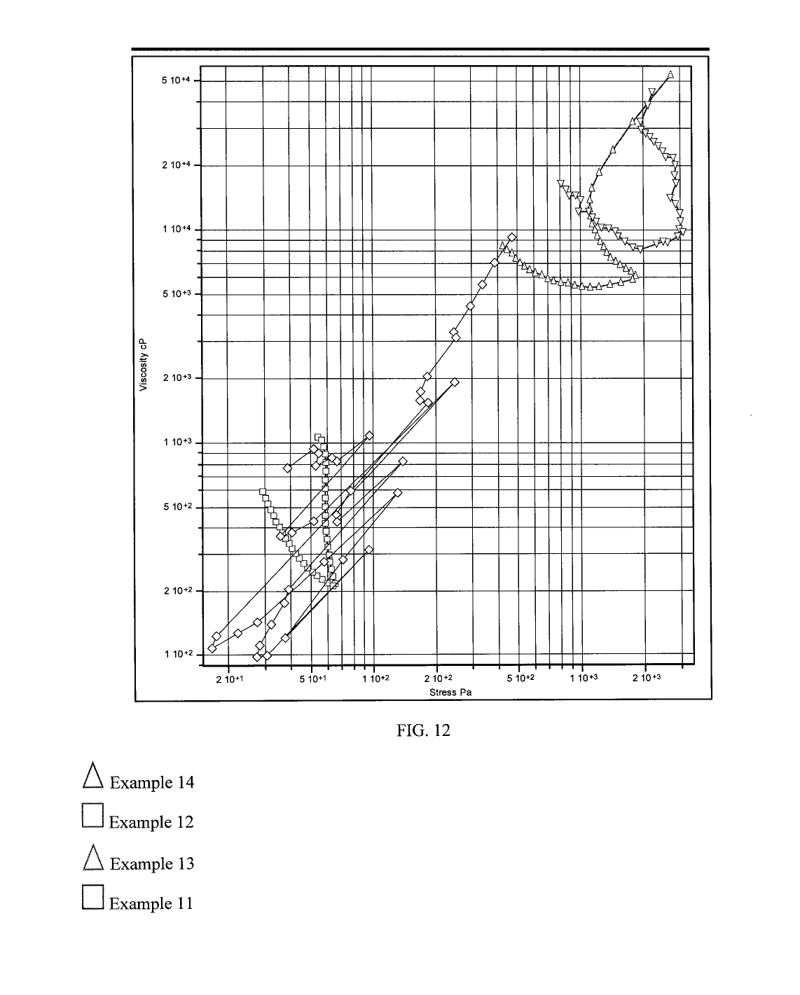

FIG. 5 is a graphical representation of the effect of various compositions on the irritation response of cells treated with known irritants, as quantified by measuring IL-8 secretion, and on cell viability. FIG. 6 is a graphical representation of Involucrin Expression—Keratinocytes (KC) differentiation. FIG. 7 is a graphical representation of PPARδ expression—ceramides related biomarker. FIG. 8 is a graphical representation of ABCA12 expression—fatty acid related biomarker. FIG. 9 is a graphical representation of DSC1 expression—cell-cell junction biomarker. FIG. 10 is a graphical representation of APQ3 expression—skin water channel.

IL-8 ELISA

Interleukin 8 (IL-8) is a chemokine and proinflammatory cytokine produced by macrophages and other cell types such as epithelial cells. It is secreted from keratinocytes in skin in response to inflammatory stimuli. IL-8 is secreted and is an important mediator of the immune reaction in the innate immune system response. IL-8 overexpressed is a biomarker of skin irritation.

For Control A, human dermal keratinocytes are left untreated. No irritation is expected, and therefore Control A provides a baseline. For Control B, IL-8 is induced in human dermal keratinocytes by applying a surfactant mixture that is a combination of sodium laureth sulfate and polyquaternium-10. For all other samples, the human dermal keratinocytes are co-treated with the surfactant mixture and a composition containing the ingredient of interest. Decreased Il-8 expression reflects the ingredient's anti-irritation activity.

In order to carry out the test method, an assay kit was employed that was obtained from R&D Systems: Human CXCL8/IL-8 Duoset ELISA Development Kit.

The following steps were followed: 1. Coat EIA high binding 96-well plate with IL-8 capture antibody overnight at room temperature 2. Prepare all reagents, standard dilutions, and samples. Warm up to room temperature. 3. Aspirate and wash the coated plate with 350 μl/well of washing buffer 4 times, then adding 300 μl/well of blocking solution incubating 1 hour at room temperature. 4. Repeat Aspirate and wash (4 times) step. 5. Add 100 μL of Standard, control, or sample to each well. Cover with a plate sealer, and incubate at room temperature for 2 hours. 6. Aspirate each well and wash, repeating the process 3 times for a total of 4 washes. 7. Add 100 μL of detection IL-8 antibody to each well. Cover with a new plate sealer, and incubate at room temperature for 2 hour. 8. Aspirate and wash 4 times. 9. Add 100 μL Biotin-Strepavidin conjugate to each well, incubating 20 minutes at room temperature. 10. Aspirate and wash 4 times. 11. Add 100 μl substrate Solution to each well. Incubate at room temperature for 20 minutes, making sure to protect the wells from the light. 12. Add 50 μL of Stop Solution to each well. Data collected using a colorimeter, absorbance was measured at 450 nanometers (nm) within 30 minutes. Wavelength correction was set to 570 nm.

MTT Assay

The MTT assay is a colorimetric assay for assessing cell viability, cell proliferation, and/or cytotoxicity. NAD(P)H-dependent cellular oxidoreductase enzymes may, under defined conditions, reflect the number of viable cells present. These enzymes are capable of reducing the tetrazolium dye MTT 3-(4,5-dimethylthiazol-2-yl)-2,5-diphenyltetrazolium bromide to insoluble formazan, which has a purple color. MTT assay can also be used to measure cytotoxicity (loss of viable cells) or cytostatic activity (shift from proliferative to resting status) of potential medicinal agents and toxic materials.

Controls A and B described above for the IL-8 Assay were also employed in this test. The mitigating effect of the test samples on the effect of Control B on the keratinocytes was measured. More specifically, while Control B has a negative effect on cell viability, cell proliferation, and/or cytotoxicity, this mitigation of this negative effect was determined by measuring the reduction of MTT.

The following steps were followed; once the liquid was removed from the wells for the IL-8 Assay described above, 100 μl/well of 0.5 mg/ml of MTT in phenol red-free DMEM (cell culture medium) was added into each of the 96-well plates. After incubating 1 hour at 37° C., all liquid was removed (MTT solution) from the wells of the culture plate. Then 100 μl of DMSO was added to each well to completely dissolve the purple product. Absorption was measured using a plate reader at 550 nm wavelength.

Cell-Cell Junction

Tight Junctions are the closely associated areas of two cells whose membranes join together forming a virtually impermeable barrier to fluid. A desmosome is a cell structure specialized for cell-to-cell adhesion. A type of junction complex, they are localized spot-like adhesions randomly arranged on the lateral sides of plasma membranes. Desmosomes are molecular complexes of cell adhesion proteins and linking proteins that attach the cell surface adhesion proteins to intracellular keratin cytoskeletal filaments.

The cell adhesion proteins of the desmosome, desmoglein (DSG) and desmocollin (DSC), are members of the cadherin family of cell adhesion molecules. They are biomarker of skin tight junctions. In particular, DSG1 is a biomarker for cell binding, the higher the expression, the better skin cell-cell junction and the better skin barrier function will be. DSC3 is a protein in humans that is encoded by the DSC3 gene, the higher the expression, the better skin cell-cell junction and the better skin barrier function will be.

In the present method, keratinocytes were treated with the sample compositions in a 6-well plate overnight. After washing with cold phosphate-buffered saline (PBS), total RNAs were prepared from each well. Real-Time Quantitative Reverse Transcription PCR (qRT-PCR) was performed to detect the target genes (DSC1 and DSG3) expression level using a One-step TaqMan® RT-PCR kit (Life Technologies).

### Test Results

### Preparation of Test Compositions

Water was heated to 180° F. glyceryl stearate, Oliwax LC, OliveM 1000 were added with stirring. Heat was held at 180° F. for 15 minutes. The mixture was allowed to slowly cool to about 125° F., and then olive oil and an emollient (INCI name Diheptyl Succinate (and) Capryloyl Glycerin/Sebacic Acid Copolymer, available as LexFeel Natural) was added. When the mixture cooled to below 100° F., the oligopeptides Adyfyline and Delisens, and a preservative blend were added. The mixture was mixed until homogenous (about 20 to 30 minutes).

A base lotion composition was prepared according to the method described above, except omitting the Adifyline and Delisens. The base lotion is denoted as Example 7. Example 8 was prepared according to the method described above, except omitting the Delisens. Example 8 contained 1 wt. % Adifyline. Example 9 was prepared according to the method described above, except omitting the Adifyline. Example 9 contained 1 wt. % Delisens. Example 10 was prepared according to the method described above, and contained 1 wt. % each of Adifyline and Delisens. Acetyl hexapeptide-46 was obtained from Lipotec under the tradename Delisens™. Delisens™ is a proprietary blend of butylene glycol, water, citric acid and acetyl hexapeptide-46, containing 0.025 wt. % acetyl hexapeptide-46. Acetyl hexapeptide-38 was obtained from Lipotec under the tradename Adifyline™. Adifyline™ is a proprietary blend of butylene glycol, water and acetyl hexapeptide-38, containing 0.05 wt. % acetyl hexapeptide-38.

The samples were diluted in water and then tested for IL-8 secretion as described in the test method above. That is, for Control A, human dermal keratinocytes were left untreated. For Control B, IL-8 a surfactant mixture that was a combination of sodium laureth sulfate and polyquaternium-10. For all other samples, the human dermal keratinocytes were co-treated with the surfactant mixture and a composition containing the ingredient of interest. Decreased Il-8 expression reflects the ingredient's anti-irritation activity. The results are summarized in Table 6 and shown graphically in FIG. 5.

TABLE 6

| | | | IL-8 | |
|---|---|---|---|---|
| | | | Inhibition | |
| Example # | Component | Dilution | (%) | MTT |
| Control A | Medium | — | 100% | 100% |
| Control B | PMA | — | 0.0% | 73% |
| Example 7 | Base Lotion | 1/100 | 64% | 89% |
| Example 8 | Base Lotion + 1 wt. % | 1/100 | 84% | 84% |
| | Adifyline (5 ppm acetyl | | | |
| | hexapeptide-38) | | | |
| Example 9 | Base Lotion + 1 wt. % | 1/100 | 116% | 83% |
| | Delisens (2.5 ppm acetyl | | | |
| | hexapeptide-46) | | | |
| Example 10 | Base Lotion + 1 wt. % | 1/100 | 48% | 86% |
| | Adifyline (5 ppm acetyl | | | |
| | hexapeptide-38) and 1 wt. | | | |
| | % Delisens (2.5 ppm | | | |
| | acetyl hexapeptide-46) | | | |

From FIG. 5 and Table 6, it can be seen that although the acetyl hexapeptides didn't individually show any significant anti-irritation activity and even slightly caused irritation, in certain circumstances, the combination showed surprising improvement.

Skin Barrier

An in vitro model was employed, using monolayer human dermal keratinocytes culture (KGM). Two controls were tested: the medium and inflammatory cytokines interleukin (IL-1b).

Skin Barrier Biomarkers: ABCA12, Involucrin, PPARδ. Different concentrations of ingredients were used to treat the keratinocyte for 24 hours. Cells were collected and total RNA prepared from the treated cells. Real-time RT-PCT was used to detect different barrier function related biomarker's gene expression level. Comparative Benchmark: Vitamin D3 (cholecalciferol) in three different concentrations.

Peroxisome proliferator-activated receptors (PPARs) are ligand activated nuclear receptors. Three PPAR subtypes have been identified: alpha, delta and gamma.

ABCA12 belongs to a group of genes called the ATP-binding cassette family, which makes proteins that transport molecules across cell membranes.

Involucrin is a protein component of human skin and in humans is encoded by the IVL gene. In binding the protein loricrin, involucrin contributes to the formation of a cell envelope that protects corneocytes in the skin. Involucrin is a highly reactive, soluble, transglutaminase substrate protein present in keratinocytes of epidermis and other stratified squamous epithelia. It first appears in the cell cytosol, but ultimately becomes cross-linked to membrane proteins by transglutaminase thus helping in the formation of an insoluble envelope beneath the plasma membrane functioning as a glutamyl donor during assembly of the cornified envelope.

Involucrin is synthesised in the stratum spinosum and cross linked in the stratum granulosum by the transglutaminase enzyme that makes it highly stable. Thus it provides structural support to the cell, thereby allowing the cell to resist invasion by micro-organisms.

Results are shown in the Tables below.

TABLE 7

| Example # | Dilution | Involucrin (%) | PPARd % | ABCA12 % |
|---|---|---|---|---|
| KGM | — | 100% | 100% | 100% |
| Example 7 | 1/1000 | 191% | 135% | 196% |
| Example 8 | 1/1000 | 211% | 162% | 240% |
| Example 9 | 1/1000 | 96% | 99% | 93% |
| Example 10 | 1/100 | 353% | 180% | 334% |

From FIG. 6 and Table 7, it can be seen that although the acetyl hexapeptides didn't individually show any significant promotion of KC differentiation, the combination showed surprising improvement.

From FIG. 7 and Table 7, it can be seen that only the combination of acetyl hexapeptides showed significant stimulation of PPARd expression in KC. Example 3 appeared to reduce PPARd expression in KC.

From FIG. 8 and Table 7, it can be seen that the combination of acetyl hexapeptides show a significant increase in ABCA12 expression in KC, while the individual samples seemed to inhibit ABCA 12 expression in KC.

TABLE 8

| Example # | Dilution | DSC1 (%) | APQ3 % |
|---|---|---|---|
| KGM | — | 100% | 100% |
| Example 7 | 1/1000 | 279% | 625% |
| Example 8 | 1/1000 | 406% | 674% |
| Example 9 | 1/1000 | 200% | 33% |
| Example 10 | 1/100 | 623% | 1030% |

From FIG. 9, it can be seen that the combination of acetyl hexapeptides significantly stimulated DSC1 expression to improve skin cell junction.

From FIG. 10, it can be seen that, although acetyl hexapeptide-46 does not individually stimulate APQ3 expression in KC, the combined acetyl hexapeptides show surprising improvement.

It can be seen that overall, the composition containing a combination of acetyl hexapeptides has the best efficacy in anti-irritation, skin barrier (KC differentiation, lipids production and cell-cell junction) and skin hydration (water channel).

Competitive Rheology Profile of Barrier Creams

Yield Stress Point was used to determine the amount of shear stress needed to initiate flow, relating to the ability to both pump and spread a product.

Example 11 was a composition according to the present invention. More specifically, the Example 11 contained glyceryl stearate, cetearyl olivate, sorbitan olivate, cetyl palmitate, sorbitan palmitate, olive oil, heptyl undecylenate, butylene glycol, acetyl hexapeptide-38, citric acid, acetyl hexapeptide-49, phenoxyethanol, and ethylhexyl glycerin.

Example 12 was a commercially available lotion containing aloe barbadensis leaf juice, ascorbic acid, ascorbyl palmitate, c12-c13 pareth-3, c12-c13 pareth-23, carthamus tinctorius seed oil, cetyl dimethicone, cholecalciferol, citric acid, citrus aurantium dulcis peel oil, citrus grandis peel oil, citrus tangerina peel oil, cyclopentasiloxane, diazolidinyl urea, dimethiconol, divinyldimethicone/dimethicone copolymer, glycine, hydroxytyrosol, l-proline, l-taurine, methylparaben, methylsulfonylmethane, n-acetyl-l-cysteine, niacinamide, olea europaea fruit oil, peg-8, peg/ppg-18/18 dimethicone, propylene glycol, propylparaben, pyridoxine hcl, retinyl palmitate, sodium chloride, tocopherol, vanillin, water, and zea mays oil, sold under the tradename Nutrashield™ by Medline Industries, Inc.

Example 13 was a commercially available ointment containing 0.44 wt. % menthol, 20.6 wt. % zinc oxide, and also containing calamine, chlorothymol, glycerin, lanolin, phenol, sodium bicarbonate, and thymo, sold under the tradename Calmoseptine™ by Calmoseptine, Inc.

Example 14 was a commercially available lotion containing 3.5 wt. % calamine, 0.2 wt. % menthol, 69 wt. % white petrolatum, 20 wt. % zinc oxide, and also containing Aloe Barbadensis Leaf Juice, Ascorbic Acid, Ascorbyl Palmitate, Carthamus Tinctorius (Safflower) Seed Oil, Cholecalciferol, Citric Acid, Citrus Aurantium Dulcis Peel Oil, Citrus Grandis Peel Oil, Glycine, Helianthus Annuus (Sunflower) Seed Oil, Hydroxytyrosol, L-proline, L-taurine, Methylparaben, Modified Corn Starch, Methylsulfonymethane, N-acetyl-L-cysteine, Niacinamide, Olea Europaea (Olive) Fruit Oil, PEG-8, Pyridoxine Hydrochloride, Retinyl Palmitate, Tapioca Starch Polymethylsilsesquioxane, Tocopherol, Vanillin, and Zea Mays (Corn) Oil, and sold under the tradename Calazime™ by Medline Industries, Inc.

The Yield Stress Point comparison for Examples 11-14 is shown in FIG. 13. It can be seen that Example 11 requires about 100 pascals (Pa) to initiate flow. Once flow has started, the product is very shear-sensitive and needs very little additional stress to keep the lotion flowing. The lotion is easy to spread, compared to Examples 12-14.

In comparison, Example 12 (Nutrashield), requires slightly more than 10 Pa of stress to start the product to flow, but is not as shear-sensitive as Example 11. In order to spread the lotion of Example 12, consistently increased stress is required.

The Yield Stress data for Examples 13 (Calmoseptine) and 14 (Calazime) indicate that these products are very viscous and require almost 10 times the amount of stress in order to initiate spreading. Additionally, Example 13 (Calmoseptine) requires consistently increased stress to continue applying this product. The Yield Stress assessment of Example 14 (Calazime) indicates that this product has a stress transition phase. Once the product has reached about 5000 Pa, the viscosity increases, showing that it also has shear thickening properties. As stress is increased, in order to spread this product, it becomes tacky and is less spreadable.

Thixotropy

A thixotropic comparison of Examples 11-14 is shown in FIG. 14 It can be seen that Example 11 becomes less viscous when very little stress is applied, and when stress is removed, the product recovers quickly to its original, viscous state.

Thixotropic assessment of Example 12 (Nutrashield) indicates that once stress is applied, the sample is extremely shear sensitive. When stress is applied, the product has no elasticity, and does not return to original state, but is irreversibly altered.

Thixotropic assessment of Examples 13 (Calmoseptine) and 14 (Calazime) indicates that the products are very viscous and require relatively high amounts of stress to start flow. Once stress is applied and then removed, the samples have a very slow recovery and may not return to the original starting state. This is evidenced by the about 80% reduction in viscosity of the Examples 13 and 14, compared to the about 40% reduction in viscosity for Example 11.

Various modifications and alterations that do not depart from the scope and spirit of this invention will become apparent to those skilled in the art. This invention is not to be duly limited to the illustrative embodiments set forth herein.

We claim:

1. A composition comprising two or more oligopeptides in a cosmetically or pharmaceutically acceptable carrier, wherein the composition includes a liquid crystal network.

2. The composition of claim 1, wherein at least one of the oligopeptides is independently selected from the group consisting of acetyl hexapeptides.

3. The composition of claim 1, wherein the least two oligopeptides are independently selected from the group consisting of acetyl hexapeptide-1, acetyl hexapeptide-7, acetyl hexapeptide-8, acetyl hexapeptide-19, acetyl hexapeptide-20, acetyl hexapeptide-22, acetyl hexapeptide-24, acetyl hexapeptide-30, acetyl hexapeptide-31, acetyl hexapeptide-37, acetyl hexapeptide-38, acetyl hexapeptide-39, and acetyl hexapeptide-46, acetyl hexapeptide-49.

4. The composition of claim 1, wherein the amount of each oligopeptide is selected to be from about 0.06 to about 100 ppm, based upon the total weight of the composition.

5. The composition of claim 1, wherein the carrier comprises a vegetal wax, olive oil, and at least one derivative or extract of olives.

6. The composition of claim 5, wherein the carrier comprises from about 0.5 to about 3 wt. % of a vegetal wax, based upon the total weight of the composition.

7. The composition of claim 5, wherein the derivative of olives includes a cetearylic ester derivative, a sorbitan ester derivative, or a combination thereof.

8. The composition of claim 7, wherein the cetearylic ester comprises cetearyl olivate and sorbitan olivate.

9. The composition of claim 1, wherein the composition further comprises from about 0.1 to about 10 wt. % of an emulsifier, based upon the total weight of the composition.

10. The composition of claim 1, wherein the carrier comprises:
a vegetal wax comprising cetyl palmitate, sorbitan palmitate and sorbitan olivate;
at least one derivative or extract of olives, selected from the group consisting of cetearylic ester derivatives, a sorbitan ester derivatives, and blends thereof; olive oil; and at least one emulsifier selected from the group consisting of glycerol esters.

11. The composition of claim 1, wherein the composition exhibits a first viscosity under static conditions, a second, reduced viscosity when shear force is applied, and then, when the shear force is removed, the viscosity of the composition returns to the first viscosity, or to a viscosity that is substantially the same as the first viscosity.

12. The composition of claim 11, wherein the second viscosity is at least 25% of the first viscosity, when a stress of up to about 100 Pa is applied as described above.

13. The composition of claim 1, wherein the composition is characterized by a yield stress point of less than about 1000 Pa, at 1 s⁻¹ at about 20° C.

14. A method for the treatment of the skin comprising the step of contacting the skin with an effective amount of a composition comprising two or more oligopeptides in a cosmetically or pharmaceutically acceptable carrier, wherein the composition includes a liquid crystal network.

15. The method of claim 14, wherein the carrier comprises:
a vegetal wax comprising cetyl palmitate, sorbitan palmitate and sorbitan olivate;
at least one derivative or extract of olives, selected from the group consisting of cetearylic ester derivatives, a sorbitan ester derivatives, and blends thereof; and olive oil.

16. The method of claim 14, wherein at least one of the oligopeptides is independently selected from the group consisting of acetyl hexapeptides.

17. The method of claim 14, wherein the least two oligopeptides are independently selected from the group consisting of acetyl hexapeptide-1, acetyl hexapeptide-7, acetyl hexapeptide-8, acetyl hexapeptide-19, acetyl hexapeptide-20, acetyl hexapeptide-22, acetyl hexapeptide-24, acetyl hexapeptide-30, acetyl hexapeptide-31, acetyl hexapeptide-37, acetyl hexapeptide-38, acetyl hexapeptide-39, and acetyl hexapeptide-46, acetyl hexapeptide-49.

18. The method of claim 14, wherein the composition further comprises from about 0.1 to about 10 wt. % of an emulsifier, based upon the total weight of the composition.

19. The method of claim 14, wherein the composition exhibits a first viscosity under static conditions, a second, reduced viscosity when shear force is applied, and then, when the shear force is removed, the viscosity of the composition returns to the first viscosity, or to a viscosity that is substantially the same as the first viscosity.

20. A method for improving the barrier function of skin, the method comprising the step of contacting the skin with an effective amount of a composition comprising two or more oligopeptides in a cosmetically or pharmaceutically acceptable carrier, wherein the composition includes a liquid crystal network.
